(19) United States
(12) Patent Application Publication
(10) Pub. No.: US 2025/0090576 A1
(43) Pub. Date: Mar. 20, 2025

(54) METHODS FOR TREATING NONTUBERCULOUS MYCOBACTERIA DISEASES

(71) Applicant: Microbion Corporation, Bozeman, MT (US)

(72) Inventor: Brett Hugh James BAKER, Bozeman, MT (US)

(21) Appl. No.: 18/727,413

(22) PCT Filed: Jan. 10, 2023
(86) PCT No.: PCT/US2023/060392

§ 371 Date: Jul. 9, 2024

Related U.S. Application Data
(60) Provisional application No. 63/298,124, filed on Jan. 10, 2022.

(51) Int. Cl. A61K 33/245; A61K 9/00; A61K 45/06; A61P 31/04

(52) U.S. Cl. CPC A61K 33/245; A61K 9/007; A61K 45/06; A61P 31/04

(57) ABSTRACT

The present disclosure provides bismuth-thiol (BT) compositions and methods for treating nontuberculous Mycobacterium infections and associated conditions in a subject in need thereof.

### CROSS-REFERENCE TO RELATED APPLICATIONS

This application claims to benefit of and priority to U.S. Provisional Application No. 63/298,124, filed Jan. 10, 2022, which is incorporated herein by reference in its entirety.

### BACKGROUND

The incidence of pulmonary infections by nontuberculous mycobacteria (NTM), such as M. avium and M. abscessus, is increasing and treatment options are limited. About 80% of pulmonary NTM is associated with M. avium. These infections are common in patients with chronic lung conditions, such as cystic fibrosis, and are associated with severe respiratory diseases. Currently accepted macrolide (clarithromycin) or azalide (azithromycin) therapies often lead to resistance or are associated with side-effects related to the duration of treatment, which can be 18-24 months and include a minimum of three antibiotics. Treatments outcomes with M. abscessus infections are even more problematic, as the cure rate in patients with pulmonary infection is only 25-58%. M. abscessus is thus referred to as the ‘incurable nightmare’.

Owing to the poor treatment outcomes and lengthy treatment duration accompanied by drug toxicity, there is an urgent medical need to develop more-effective and safe regimens consisting of drugs with potent anti-NTM activity.

The present disclosure addresses these and other issues related to the treatment of NTM disease.

### SUMMARY

In some embodiments, the present disclosure provides a method of treating an infection in a subject caused by nontuberculous Mycobacterium (NTM), the method comprising administering to the subject an effective amount of a bismuth-thiol (BT) composition that comprises a BT compound.

In some embodiments, the present disclosure provides a method of reducing NTM intracellular bacterial burden in a subject, comprising contacting an infected cell of the subject with an effective amount of a bismuth-thiol (BT) composition that comprises a BT compound.

In some embodiments, the present disclosure provides a method for treating or providing prophylaxis against a nontuberculous Mycobacterium (NTM) lung infection in a subject in need of treatment or prophylaxis, comprising: administering to the lungs of the subject for an administration period, a BT composition comprising a BT compound.

In some embodiments, the present disclosure provides a method for treating a biofilm-associated nontuberculous Mycobacterium (NTM) infection in the lungs of a subject in need thereof, comprising administering to the subject a BT composition comprising a BT compound.

In some embodiments, the NTM infection is a pulmonary infection. In some embodiments, the NTM infection is an extrapulmonary infection. In some embodiments, the NTM infection is a chronic pulmonary infection.

In some embodiments, the NTM infection is located in or on the lung mucosa, the bronchi, the alveoli, the macrophages, and/or the bronchioles. In some embodiments, the NTM infection is a cutaneous NTM infection. In some embodiments, the NTM infection is an infection of the skin, bones, joints, lymphatic system, and/or soft tissue. In some embodiments, the NTM infection is located in the macrophages. In some embodiments, the NTM infection is at least partially located in the macrophages. In some embodiments, the macrophage cells are THP-1 cells. In some embodiments, the NTM infection is located in the histiocytes.

In some embodiments, the present disclosure provides a method of treating an NTM infection in a subject, the method comprising: (i) testing for the presence of bacteria-infected macrophages in a biological sample from the subject; and (ii) administering an effective amount of the bismuth-thiol (BT) composition that comprises a BT compound to the subject if the sample tests positive for bacteria-infected macrophages.

In some embodiments, the present disclosure provides a method of treating an NTM infection in a subject having a macrophage infection, comprising administering to the subject an effective amount of a bismuth-thiol (BT) composition that comprises a BT compound.

In some embodiments, the method further comprises testing for the presence of bacteria-infected macrophages in a biological sample from the subject, and administering an effective amount of the bismuth-thiol (BT) composition to the subject if the sample tests positive for bacteria-infected macrophages.

In some embodiments, the NTM infection is caused by M. avium, M. avium subsp. hominissuis (MAH), M. abscessus, M. chelonae, M. bolletii, M. kansasii, M. chimaera, M. ulcerans, M. avium complex (MAC) (M. avium and M. intracellulare), M. conspicuum, M. kansasii, M. peregrinum, M. immunogenum, M. xenopi, M. marinum, M. malmoense, M. massiliense, M. mucogenicum, M. nonchromogenicum, M. porcinum, M. scrofulaceum, M. simiae, M. smegmatis, M. szulgai, M. terrae, M. terrae complex, M. haemophilum, M. genavense, M. asiaticum, M. shimoidei, M. gordonae, M. nonchromogenicum, M. triplex, M. lentiflavum, M. celatum, M. fortuitum, M. fortuitum complex (M. fortuitum and M. chelonae), or a combination thereof. In some embodiments, the NTM infection is caused by M. avium or M. abscessus. In some embodiments, the NTM infection is caused by M. avium complex (MAC) (M. avium and M. intracellulare). In some embodiments, the NTM infection is caused by M. abscessus subsp. abscessus, M. abscessus subsp. bolletti, or M. abscessus subsp. massiliense.

In some embodiments, the NTM infection is resistant to standard-of-care antibacterial drug treatment. In some embodiments, the NTM infection is resistant to amikacin. In some embodiments, the NTM infection is resistant to macrolide or azalide therapy.

In some embodiments, the BT compound is selected from the group consisting of BisBAL, BisEDT, Bis-dimercaprol, BisDTT, Bis-2-mercaptoethanol, Bis-DTE, BisPyr, BisEry, BisTol, BisBDT, BisPDT, BisPyr/BAL, BisPyr/BDT, BisPyr/EDT, BisPyr/PDT, Bis-Pyr/Tol, BisPyr/Ery, bismuth-1-mercapto-2-propanol, BisEDT/CSTMN (1:1), BisPyr/CSTMN (1:1), BisBAL/CSTMN (1:1), BisTOL/CSTMN (1:1), and BisEDT/2-hydroxy-1-propanethiol. In some embodiments, the BT compound is BisEDT or BisBAL. In some embodiments, the BT compound is BisEDT.

In some embodiments, the BT composition is administered by inhalation. In some embodiments, the BT composition is administered to the lungs of a subject. In some embodiments, the BT composition is administered to the lungs of a subject by a nebulizer, dry powder inhalation, nanoparticle inhalation, metered-dose inhalation, or any other drug inhalation method known in the art. In some embodiments, the dry powder is micronized. In some embodiments, the nanoparticles are lipid nanoparticles. In some embodiments, the nanoparticles are dry nanoparticles. In some embodiments, the nanoparticles are lipid nanoparticles. In some embodiments, the concentration of bismuth in the lungs after a single daily dose is from about 0.03 μg/g lung tissue to about 3 μg/g lung tissue.

In some embodiments, the BT composition is administered once per month, twice per month, three times per month, four times per month, once every two weeks, once per week, twice per week, or three times per week In some embodiments, the BT composition is administered once per week, twice per week or three times per week. In some embodiments, the BT composition is administered once per week. In some embodiments, the BT composition is administered for a period of less than 24 months, less than 18 months, less than 12 months, less than 9 months, less than 6 months, less than 3 months, or less than 1 month. In some embodiments, the BT composition is administered for a period of 1 to 56 days.

In some embodiments, the subject has a chronic lung condition. In some embodiments, the chronic lung condition is cystic fibrosis, chronic pneumonia, bronchiectasis, restrictive lung disease, interstitial lung disease, pulmonary hypertension, pulmonary fibrosis, chronic obstructive pulmonary disorder (COPD), emphysema, or asthma. In some embodiments, the chronic lung condition is cystic fibrosis, chronic bronchitis, emphysema, bronchiectasis, pulmonary fibrosis, asbestosis, pneumonitis, chronic obstructive pulmonary disorder (COPD), or asthma. In some embodiments, the chronic lung condition is cystic fibrosis. In some embodiments, the subject is immunocompromised.

In some embodiments, the subject is administered about 30 μg to about 3,000 μg of BT compound per dose. In some embodiments, the subject is administered about 100 μg to about 1,000 μg of BT compound per dose.

In some embodiments, the methods further comprise administering an effective amount of an additional antibacterial agent. In some embodiments, the additional antibacterial agent is amikacin, clarithromycin, azithromycin, ethambutol, rifampicin, tigecycline, linezolid, imipenem, cefoxitin, or combination thereof. In some embodiments, the additional antibacterial agent is amikacin or clarithromycin. In some embodiments, the additional antibacterial agent is amikacin.

In some embodiments, administration of a BT composition and an additional antibacterial agent to a subject in need results in a synergistic effect in treating the NTM infection.

In some embodiments, the BT composition and the additional antibacterial agent are administered simultaneously, separately, or sequentially. In some embodiments, the BT composition is administered concurrently with, prior to, or after the additional antibacterial agent.

### BRIEF DESCRIPTION OF THE DRAWINGS

FIG. 1 shows the mycobacterial survival vs. control in THP-1 macrophages infected with M. avium strain 104 (MAH 104), M avium strain 3388 (MAH 3388), and M. avium cystic fibrosis patient strain DNA00703 after treatment with amikacin, BisEDT (soluble and insoluble formations), or combinations thereof.

FIG. 2 shows the mycobacterial survival vs. control in THP-1 macrophages infected with M. abscessus strain 19977 (B), M. abscessus cystic fibrosis patient strain 01715 (C), and M. abscessus cystic fibrosis patient strain 00703 (D) after treatment with minimum inhibitory concentrations of amikacin, BisEDT (soluble and insoluble formations), or combinations thereof.

FIG. 3A shows the transmission electron microscopy images (TEM) of uninfected THP-1 macrophages 72 h after plating (control).

FIG. 3B shows the transmission electron microscopy images of THP-1 macrophages 24 h after infection with M. avium (no BisEDT treatment).

FIG. 3C shows the transmission electron microscopy images of THP-1 macrophages 48 h after infection with M. avium (no BisEDT treatment).

FIG. 3D shows the transmission electron microscopy images of THP-1 macrophages infected with M. avium 24 h after treatment with BisEDT (BIZ).

FIG. 3E shows the transmission electron microscopy images of THP-1 macrophages 24 h after infection with M. abscessus (no BisEDT treatment).

FIG. 3F shows the transmission electron microscopy images of THP-1 macrophages infected with M. abscessus 24 h after treatment with BisEDT (BIZ).

FIG. 3G shows the transmission electron microscopy images of THP-1 macrophages infected with M. abscessus 48 h after treatment with BisEDT (BIZ).

FIG. 4 is a schematic diagram showing the inoculation, challenge, and recovery steps of the of the MBEC assay used in Example 5 to measure the susceptibility ofM. avium and M. abscessus biofilm to BisEDT.

FIG. 5A shows the biofilm biomass formed on the pegs after inoculation with M. avium by staining purpose with a crystal violet assay.

FIG. 5B shows the biofilm biomass formed on the pegs after inoculation with M. abscessus by staining purple with a crystal violet assay.

FIG. 6 shows the normalized heatmap distribution of resistance to antibiotics of Mycobacterium abscessus.

FIG. 7 provides a diagram summarizing the study groups and dose schedule for an in vivo chronic M. abscessus infection study in SCID mice. Treatment groups consisted of baseline (to determine initial infection levels), vehicle, inhaled BisEDT (200 μg/kg/day), inhaled BisEDT (1000 μg/kg/day) or inhaled amikacin (100 mg/kg/day). Treatment lasted for 28 days (6 days/week) after which the lung and spleen samples were harvested for CFU determination or histopathology.

FIG. 8A shows the colony forming units (CFU) recovered from lung homogenates in an in vivo chronic M. abscessus infection study in SCID mice.

FIG. 8B shows the colony forming units (CFU) recovered from spleen homogenates in an in vivo chronic M. abscessus infection study in SCID mice.

### DEFINITIONS

Unless defined otherwise, all technical and scientific terms used herein have the meaning commonly understood by a person skilled in the art of the present disclosure. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

“Comprise” as is used in this description and in the claims and its conjugations are used in its non-limiting sense to mean that items following the word are included, but items not specifically mentioned are not excluded. The present disclosure may suitably “comprise”, “consist of”, or “consist essentially of”, the steps, elements, and/or reagents described in the claims.

Unless specifically stated or obvious from context, as used herein, the term “or” is understood to be inclusive. Unless specifically stated or obvious from context, as used herein, the terms “a”, “an”, and “the” are understood to be singular or plural.

Throughout the present specification, the terms “about” and/or “approximately” may be used in conjunction with numerical values and/or rages. The term “about” is understood to mean those values near to a recited value. For example, “about 40 [units]” can mean within +10% of 40 (e.g., from 36 to 44), within ±9%, ±8%, ±7%, ±6%, ±5%, ±4%, ±3%, ±2%, +1%, less than +1%, or any other value or range of values therebetween. Furthermore, the phrases “less than about [a value]” or “greater than about [a value]” should be understood in view of the definition of the term “about” provided herein. The terms “about” and “approximately” may be used interchangeably.

The term “bismuth” refers to the 83^{rd} element of the periodic table, or atoms or ions thereof. Bismuth can occur in the metallic state or in the ionized state, such as in the III or V oxidation state. Bismuth ions can form complexes with anions, either to make bismuth salts, or to form complex anions which are then further complexed with one or more additional cation(s). Bismuth can also form covalent bonds to other atoms, such as sulfur.

As disclosed herein, a “bismuth-thiol compound” or “BT compound” is a compound that has a bismuth atom covalently bound to one, two or three other sulfur atoms present on one or more thiol compounds. The term “thiol” refers to a carbon-containing compound, or fragment thereof, containing an —SH group and can be represented by the general formula R—SH. These thiol compounds include compounds with one, two, three or more S atoms. Thiol compounds can have other functionality, such as alkyl, hydroxyl, carbocyclyl, heterocyclyl, aryl, heteroaryl, amino, and other substituents. Thiol compounds having two or more S atoms can chelate the bismuth atom, such that two S atoms from the same molecule covalently bond with the bismuth atom. Exemplary bismuth-thiol compounds are shown below:

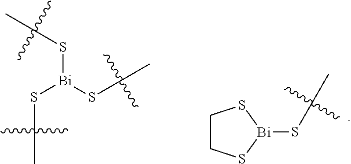

The term “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. Preferred subjects are humans.

As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week.

“Coadministration” refers to the administration of the two agents in any manner in which the pharmacological effects of both agents are manifest in the patient at the same time. Thus, concomitant administration does not require that a single pharmaceutical composition, the same dosage form, or even the same route of administration be used for administration of both agents or that the two agents be administered at precisely the same time. However, in some situations, coadministration will be accomplished most conveniently by the same dosage form and the same route of administration, at substantially the same time.

As used herein, the term “in combination” or “in further combination” or “further in combination” refers to the use of an additional prophylactic and/or therapeutic agent as well as a BT composition of the present disclosure. The use of the term “in combination” does not restrict the order in which prophylactic and/or therapeutic agents are administered to a subject. A first prophylactic or therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent (different from the first prophylactic or therapeutic agent) to a subject.

As used herein, a therapeutic agent that “prevents” a disorder or condition refers to a compound that, in a statistical sample, reduces the occurrence of the disorder or condition in the treated sample relative to an untreated control sample, or delays the onset or reduces the severity of one or more signs and symptoms of the disorder or condition relative to the untreated control sample.

As used herein, the terms “prophylactic agent” and “prophylactic agents” refer to an agent, such as a BT composition of the present disclosure, which can be used in the prevention, management, or control of one or more signs and symptoms of a disease or disorder, in particular, a disease or disorder associated with a microbial (e.g. bacterial and/or fungal) infection, such as diabetic foot infection.

The term “treating” means one or more of relieving, alleviating, delaying, reducing, improving, or managing at least one symptom of a condition in a subject. The term “treating” may also mean one or more of arresting, delaying the onset (i.e., the period prior to clinical manifestation of the condition) or reducing the risk of developing or worsening a condition.

The term “managing” includes therapeutic treatments as defined above. Managing includes achieving a steady state level of infection as determined by known methods in the art. The steady state can include evaluation of one or more of the severity of the infection(s), the size and location of the infection(s), the number of different microbial pathogens present in the infection(s), the level of antibiotic tolerant or resistant microbial pathogens, the degree of response to treatment, such as with a BT composition disclosed herein, the degree of biofilm formation and reduction, and the side effects experienced by the subject. During management of an infection, the infection may fluctuate from increasing to lessening in severity, in the amount or extent of infection, amount of side effects experienced by the subject, or other subject outcome indicia. Over a period of time, such as days, month, or years, the degree of management of the infection can be determined by evaluation of the above factors to assess whether the clinical course of infection has improved, is bacteriostatic, or has worsened. In some embodiments, managing an infection include successful treatment of microbial pathogen(s) that are otherwise drug tolerant or drug resistant.

The term “lessen the severity” of infection(s) refers to an improvement in the clinical course of the infection on any measurable basis. Such basis can include measurable indices such as reducing the extent of infection(s), whether the infection(s) are considered acute, the number and identity of microbial pathogens causing the infection(s), the extent/spread/amount of microbial (e.g. bacterial and/or fungal) biofilms, and side effects experienced by the subject. In some embodiments, lessening the severity of an infection is determined by measuring an improvement in clinical signs and symptoms of infection. In some embodiments, lessening the severity involves halting a steady decline in outcome to achieve stabilized infection(s), resulting in the subject entering successful management of the infection(s). In other embodiments, lessening the severity can result in substantial to complete treatment of the infection(s).

In some embodiments, lessening the severity of infections and/or symptoms can relate to patient-reported outcomes (“PROs”). A PRO instrument is defined as any measure of a subject's health status that is elicited from the patient and determines how the patient “feels or functions with respect to his or her health condition.” PROs are particularly useful in reporting outcomes in DFI and whether the severity of symptoms has been reduced or lessened. Such symptoms can be observable events, behaviors, or feelings (e.g., ability to walk quickly, lack of appetite, expressions of anger), or unobservable outcomes that are known only to the patient (e.g., perceptions of pain, feelings of depression). In some embodiments, lessening the severity of infections and/or symptoms can be determined by physician assessments commonly known in the art, for example by an 8 item wound score.

An “effective amount”, as used herein, refers to an amount that is sufficient to achieve a desired biological effect. A “therapeutically effective amount”, as used herein refers to an amount that is sufficient to achieve a desired therapeutic effect. For example, a therapeutically effective amount can refer to an amount that is sufficient to improve at least one sign or symptom of an infection.

The phrase “pharmaceutically acceptable” is employed herein to refer to those compounds, materials, compositions, and/or dosage forms which are, within the scope of sound medical judgment, suitable for use in contact with the tissues of a subject without excessive toxicity, irritation, allergic response, or other problem or complication, commensurate with a reasonable benefit/risk ratio.

A “response” to a method of treatment can include, among other things, a decrease in or amelioration of negative signs and symptoms, a decrease in the progression of an infection or symptoms thereof, an increase in beneficial symptoms or clinical outcomes, a lessening of side effects, stabilization of the infection, and partial or complete remedy of infection, partial or full wound closure, reduction in wound size, or complete or substantially complete re-epithelialization, among others.

“Antibiotic susceptibility or sensitivity” refers to whether a bacteria will be successfully treated by a given antibiotic. Similarly, “Antifungal susceptibility or sensitivity” refers to whether a fungi will be successfully treated by a given antibiotic. Testing for susceptibility can be performed by methods known in the art such as the Kirby-Bauer method, the Stokes method and Agar Broth dilution methods. The effectiveness of an antibiotic in killing the bacteria or preventing bacteria from multiplying can be observed as areas of reduced or stable amount, respectively, of bacterial growth on a medium such as a wafer, agar, or broth culture.

“Antimicrobial tolerance” refers to the ability of a microbe, such as bacteria or fungi, to naturally resist being killed by antibiotics. It is not caused by mutant microbes but rather by microbial cells that exist in a transient, dormant, non-dividing state. Antibiotic or drug tolerance is caused by a small subpopulation of microbial cells termed persisters. Persisters are not mutants, but rather are dormant cells that can survive the antimicrobial treatments that kill the majority of their genetically identical siblings. Persister cells have entered a non- or extremely slow-growing physiological state which makes them insensitive (refractory or tolerant) to the action of antimicrobial drugs. Similarly, “antibiotic tolerance” refers to the ability of a bacteria to naturally resist being killed by antibiotics and “antifungal tolerance” refers to the ability of a fungi to naturally resist being killed by antibiotics.

“Antimicrobial resistance” refers to the ability of a microbe to resist the effects of medication that once could successfully treat the microbe. Microbes resistant to multiple antimicrobials are called multidrug resistant (MDR). Resistance arises through one of three mechanisms: natural resistance in certain types of bacteria, genetic mutation, or by one species acquiring resistance from another. Mutations can lead to drug inactivation, alteration of the drugs binding site, alteration of metabolic pathways and decreasing drug permeability.

As used herein, the terms “antibacterial activity”, “antifungal activity” and “antimicrobial activity”, with reference to a BT composition of the present disclosure, refers to the ability to kill and/or inhibit the growth or reproduction of a particular microorganism. In certain embodiments, antibacterial or antimicrobial activity is assessed by culturing bacteria, e.g., Gram-positive bacteria (e.g., S. aureus), Gram-negative bacteria (e.g., A. baumannii, E. coli, and/or P. aeruginosa) or bacteria not classified as either Gram-positive or Gram-negative, or fungi according to standard techniques (e.g., in liquid culture or on agar plates), contacting the culture with a BT composition of the present disclosure and monitoring cell growth after said contacting. For example, in a liquid culture, bacteria may be grown to an optical density (“OD”) representative of a mid-point in exponential growth of the culture; the culture is exposed to one or more concentrations of a BT compound of the present disclosure, or variants thereof, and the OD is monitored relative to a control culture. Decreased OD relative to a control culture is representative of antibacterial activity (e.g., exhibits lytic killing activity). Similarly, bacterial colonies can be allowed to form on an agar plate, the plate exposed to a BT composition of the present disclosure, or variants thereof, and subsequent growth of the colonies evaluated related to control plates. Decreased size of colonies, or decreased total numbers of colonies, indicate antibacterial activity.

“Biofilm” refers any syntrophic consortium of microorganisms in which cells stick to each other and often also to a surface. These adherent cells become embedded within a slimy extracellular matrix that is composed of extracellular polymeric substances (EPS). Upon formation of biofilms, microbial resistance to antibiotics is up to 1000 times greater compared to that of planktonic bacteria. Bacterial aggregates are clusters of laterally aligned cells can initiate biofilm development, which has a more complex and denser 3-D structure. In some embodiments, the biofilm may comprise one or more species of bacteria (e.g., Pseudomonas aeruginosa and Staphylococcus aureus) and/or one or more different phyla (e.g., bacteria, virus and fungi).

The term “infection” is used herein in its broadest sense and refers to any infection, such as viral infection or caused by a microorganism bacterial infection, fungal infection or parasitic infection (e.g. protozoa, amoeba or helminths). Examples of such infections can be found in a number of well-known texts such as “Medical Microbiology” (Greenwood, D., Slack, R., Peutherer, J., Churchill Livingstone Press, 2002); “Mims' Pathogenesis of Infectious Disease” (Mims, C., Nash, A., Stephen, J., Academic Press, 2000); “Fields” Virology. (Fields, B N, Knipe D M, Howley, P M, Lippincott Williams and Wilkins, 2001); and “The Sanford Guide To Antimicrobial Therapy,” 26th Edition, JP Sanford et al. (Antimicrobial Therapy, Inc., 1996), which is incorporated by reference herein. The presence of infection in e.g. a diabetic foot wound is defined by clinical signs and symptoms of infection or inflammation, not by the culture of microorganisms, which are always present. However, immediately following resolution of clinical signs and symptoms of a wound infection, most patients will still have the underlying ulcer (e.g. diabetic foot ulcer), which requires continued treatment to facilitate complete wound closure. Of note, however, is that many wound specialists believe that in addition to the clinically defined state of infection, a less clinically apparent pathological state, known as “critical colonization” exists. In this state, a wound may be delayed or arrested in wound healing due to the subclinical presence of a high level of bacteria. This critical colonization, sometimes referred to as a high ‘wound bioburden’, is often polymicrobial and associated with biofilm-producing bacteria; it has been shown to induce, or prolong, the active inflammatory phase of repair, thus preventing a normal wound healing process. The bacterial cells that comprise such biofilms are difficult to recognize because they often exist in a viable, but nonculturable (VBNC), state (Pasquaroli 2013), yet they are adherent to surfaces and are typically more tolerant and resistant than their planktonic counterparts to antibiotics and antiseptics (Costerton 1999, Nguyen 2011). The term “infection” therefore contemplates the clinically defined state of infection as well as “critical colonization.”

“Airway surface” and “pulmonary surface,” as used herein, include pulmonary airway surfaces such as the bronchi and bronchioles, alveolar surfaces, and nasal and sinus surfaces.

As used herein, the term “volumetric median diameter” or “VMD” of an aerosol is the particle size diameter identified such that half of the mass of the aerosol particles is contained in particles with larger diameter than the VMD, and half of the mass of the aerosol particles is contained in particles with smaller diameter than the VMD. VMD is typically measured by laser diffraction.

“Mass median aerodynamic diameter” or “MMAD” is a measure of the aerodynamic size of a dispersed aerosol particle. The aerodynamic diameter is used to describe an aerosolized particle in terms of its settling behavior, and is the diameter of a unit density sphere having the same settling velocity, generally in air, as the particle in question. The aerodynamic diameter encompasses particle shape, density and physical size of a particle. As used herein, MMAD refers to the midpoint or median of the aerodynamic particle size distribution of an aerosolized particle determined by cascade impaction and/or laser time of flight and/or cascade impactor.

“Mass median diameter” or “MMD” is a measure of mean particle size. Any number of commonly employed techniques can be used for measuring mean particle size.

As used herein, “D90” refers to the 90% value of particle diameter (either the microparticle or aerosolized particle). For example if D90=1 μm, 90% of the particles are smaller than 1 μm. Similarly, “D80” refers to the 80% value of particle diameter (either the microparticle or aerosolized particle), “D70” refers to the 70% value of particle diameter (either the microparticle or aerosolized particle), “D60” refers to the 60% value of particle diameter (either the microparticle or aerosolized particle), “D50” refers to the 50% value of particle diameter (either the microparticle or aerosolized particle), “D40” refers to the 40% value of particle diameter (either the microparticle or aerosolized particle), “D30” refers to the 30% value of particle diameter (either the microparticle or aerosolized particle), “D20” refers to the 20% value of particle diameter (either the microparticle or aerosolized particle), “D10” refers to the 10% value of particle diameter (either the microparticle or aerosolized particle).

As used herein, “monodisperse” refers to a collection of particles (bulk or aerosol dispersion) comprising particles of a substantially uniform MMD and/or MMAD and/or VMD.

As used herein, the term “deposition efficiency” refers to the percentage of the delivered dose that is deposited into the area of interest. Thus, the deposition efficiency of a method and/or system for delivering an aerosolized medicament into the lungs is the amount by mass of the aerosol deposited into the lungs divided by the total amount of the aerosol delivered by the system to the nares.

As used herein, “substantially” or “substantial” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking, the nearness of completion will be so as to have the same overall result as if absolute and total completion were obtained. The use of “substantially” is equally applicable when used in a negative connotation to refer to the complete or near complete lack of action, characteristic, property, state, structure, item, or result. For example, a composition that is “substantially free of” other active agents would either completely lack other active agents, or so nearly completely lack other active agents that the effect would be the same as if it completely lacked other active agents. In other words, a composition that is “substantially free of” an ingredient or element or another active agent may still contain such an item as long as there is no measurable effect thereof.

### DETAILED DESCRIPTION

Nontuberculous Mycobacterium (NTM) are opportunistic pathogens, causing mostly TB-like pulmonary diseases largely in immunocompromised patients or patients with pre-existing conditions, such as cystic fibrosis (CF), chronic bronchitis, emphysema, bronchiectasis, pulmonary fibrosis, asbestosis, pneumonitis, chronic obstructive pulmonary disorder (COPD), asthma, and other underlying lung disease. The annual prevalence of NTM pulmonary disease (NTM-PD) varies in different regions, ranging from 0.2/100 000 to 9.8/100 000 1, 2 with an overall alarming growth rate 3, 4. The situation is worse among vulnerable populations. Large-scale epidemiological studies from several countries and regions reported a high prevalence of 3.3-22.6% in CF patients, whereas COPD patients treated with inhaled corticosteroid therapy are associated with a 29-fold increased risk of NTM.

Unfortunately, the development of effective drug regimens for treating NTM-PD has been a significant challenge, meaning therapeutic options are often limited and patient outcomes poor, due in part to the ability of NTMs to form biofilms, lengthy treatment regimes, and drug-related side effects.

To address these and other issues, the present disclosure provides bismuth-thiol compositions and methods for the treatment of NTM diseases and related conditions. BT compositions have been established as highly effective antimicrobial agents for treating various pathogens, including those involved in CF, wounds, and co-infection, as described in WO2020/028558, WO2020/028561, and WO2021/195236, respectively, each of which is incorporated herein by reference.

### Methods of Use

In some embodiments, the present disclosure provides a method of treating an infection in a subject caused by nontuberculous Mycobacterium (NTM), the method comprising administering to the subject an effective amount of a bismuth-thiol (BT) composition that comprises a BT compound.

In some embodiments, the BT compound is a BT compound disclosed herein. In some embodiments, the BT compound is selected from the group consisting of BisBAL, BisEDT, Bis-dimercaprol, BisDTT, Bis-2-mercaptoethanol, Bis-DTE, BisPyr, BisEry, BisTol, BisBDT, BisPDT, BisPyr/BAL, BisPyr/BDT, BisPyr/EDT, BisPyr/PDT, Bis-Pyr/Tol, BisPyr/Ery, bismuth-1-mercapto-2-propanol, BisEDT/CSTMN (1:1), BisPyr/CSTMN (1:1), BisBAL/CSTMN (1:1), BisTOL/CSTMN (1:1), and BisEDT/2-hydroxy-1-propanethiol. In some embodiments, the BT compound is BisEDT or BisBAL. In some embodiments, the BT compound is BisEDT.

In some embodiments, the NTM infection is a pulmonary infection. In some embodiments, the NTM infection is a chronic pulmonary infection. In some embodiments, the NTM infection is an extrapulmonary infection. In some embodiments, the NTM infection is a cutaneous infection.

In some embodiments, the extrapulmonary infection is an infection of the skin, bones, joints, lymphatic system, soft tissue, or combination thereof.

In some embodiments, the NTM infection is a biofilm-associated NTM infection. In some embodiments, the NTM infection comprises planktonic cells and biofilm.

In some embodiments, the NTM infection is caused by an antibiotic-resistant strain of NTM. In some embodiments, the NTM infection is resistant to standard-of-care antibacterial agents. Is some embodiments, the NTM infection is resistant to macrolide or azalide therapy. In some embodiments, the NTM infection is resistant to aminoglycoside therapy. In some embodiments, the NTM infection is resistant to amikacin, clarithromycin, azithromycin, ethambutol, rifampicin, tigecycline, linezolid, imipenem, cefoxitin, or combination thereof. In some embodiments, the NTM infection is resistant to amikacin. In some embodiments, the antibacterial agent-resistant NTM infection comprises biofilm.

In some embodiments, the NTM infection is caused by M. avium, M. avium subsp. hominissuis (MAH), M. abscessus, M. chelonae, M. bolletii, M. kansasii, M. ulcerans, M. avium complex (MAC) (M. avium and M. intracellulare), M. conspicuum, M. kansasii, M. peregrinum, M. immunogenum, M. xenopi, M. marinum, M. malmoense, M. marinum, M. mucogenicum, M. nonchromogenicum, M. scrofulaceum, M. simiae, M. smegmatis, M. szulgai, M. terrae, M. terrae complex, M. haemophilum, M. genavense, M. asiaticum, M. shimoidei, M. gordonae, M. nonchromogenicum, M. triplex, M. lentiflavum, M. celatum, M. fortuitum, M. fortuitum complex (M. fortuitum and M. chelonae), or a combination thereof. In some embodiments, the NTM infection is caused by M. abscessus, M. avium, M. intracellulare, M. fortuitum, M. gordonae, M. kansasii, M. avium complex (MAC), M. abscessus complex (MABSC) M. marinum, M. terrae and M. cheloni. In some embodiments, the NTM infection is caused by M. abscessus, M. avium, or a combination thereof. In some embodiments, the NTM infection is caused by M. abscessus, M. avium complex (M. avium and M. intracellulare), or a combination thereof. In some embodiments, the NTM lung infection is caused by M. avium complex (M. avium and M. intracellulare). In some embodiments, the M. avium is M. avium subsp. hominissuis. In some embodiments, the M. abscessus is M. abscessus subsp. abscessus, M. abscessus subsp. bolletti, or M. abscessus subsp. massiliense.

In some embodiments, the NTM infection is located in or on the lung mucosa, the bronchi, the alveoli, the macrophages, and/or the bronchioles. In some embodiments, the NTM infection is a cutaneous NTM infection. In some embodiments, the NTM infection is an infection of the skin, bones, joints, lymphatic system, and/or soft tissue. In some embodiments, the NTM infection is located in the macrophages. In some embodiments, the NTM infection is at least partially located in the macrophages. In some embodiments, the macrophage cells are THP-1 cells. In some embodiments, the macrophages are alveolar macrophages. In some embodiments, the macrophages are M1 or M1-like macrophages. In some embodiments, the macrophages are infected with one or more strains of M. abscessus and/or M. avium. In some embodiments, the macrophages are infected with one or more strains of M. abscessus and/or M. avium complex. In some embodiments, the NTM infection is located in the histiocytes. In some embodiments, the infection is located in the osteoclasts.

In some embodiments, upon administration of the BT composition to the subject, the bacterial load in the macrophages is reduced.

In some embodiments, the subject in need of treatment has a chronic disease. In some embodiments, the subject has a chronic lung condition. In some embodiments, the chronic lung condition is cystic fibrosis, chronic pneumonia, bronchiectasis, restrictive lung disease, interstitial lung disease, pulmonary hypertension, pulmonary fibrosis, chronic obstructive pulmonary disorder (COPD), emphysema, or asthma. In some embodiments, the chronic lung condition is cystic fibrosis, chronic bronchitis, emphysema, bronchiectasis, pulmonary fibrosis, asbestosis, pneumonitis, chronic obstructive pulmonary disorder (COPD), or asthma. In some embodiments, the chronic lung condition is cystic fibrosis. In some embodiments, the subject is immunocompromised.

In some embodiments, the subject having NTM disease also suffers from co-morbidities such as malignancies and/or cardiovascular diseases. In some embodiments, the co-morbidity is selected from the group consisting of diabetes, mitral valve disorder, acute bronchitis, pulmonary hypertension, pneumonia, asthma, cystic fibrosis, pulmonary fibrosis, a larynx anomaly, a trachea anomaly, a bronchus anomaly, aspergillosis, HIV, bronchiectasis, arrhythmia, cancer (including, but not limited to trachea cancer, bronchus cancer, prostate cancer, lung cancer, ovarian cancer, breast cancer, etc.), chronic heart failure, ischemic heart disease COPD, Crohn's disease, Sjogren syndrome, rheumatoid arthritis, panbronchiolitis, systemic lupus erythematosus (SLE), systemic sclerosis, dyslipidemia, gastroesophageal reflux (GERD), or a combination thereof. In some embodiments, the co-morbidity is selected from the group consisting of diabetes, mitral valve disorder, acute bronchitis, pulmonary hypertension, pneumonia, asthma, trachea cancer, bronchus cancer, lung cancer, cystic fibrosis, pulmonary fibrosis, a larynx anomaly, a trachea anomaly, a bronchus anomaly, aspergillosis, HIV, bronchiectasis, or combination thereof.

In some embodiments, the BT composition is administered to the subject in need thereof by inhalation. In some embodiments, the BT composition is administered to the subject in need thereof by inhalation of an aerosol, as described herein. In some embodiments, the BT composition is administered to the lungs of a subject. In some embodiments, the BT composition is aerosolized and administered to the lungs of a subject. In some embodiments, the BT composition is administered to the lungs of a subject by a nebulizer, dry powder inhalation, nanoparticle inhalation, metered-dose inhalation, or any other drug inhalation method known in the art. In some embodiments, the dry powder is micronized. In some embodiments, the nanoparticles are lipid nanoparticles. In some embodiments, the nanoparticles are dry nanoparticles. In some embodiments, the nanoparticles are lipid nanoparticles. In some embodiments, the concentration of bismuth in the lungs after a single daily dose is from about 0.03 μg/g lung tissue to about 3 μg/g lung tissue.

In some embodiments, the subject in need thereof is administered about 30 μg to about 3,000 μg of BT compound per dose, e.g., about 30 μg, about 100 μg, about 200 μg, about 300 μg, about 400 μg, about 500 μg, about 600 μg, about 700 μg, about 800 μg, about 900 μg, about 1000 μg, about 1100 μg, about 1200 μg, about 1300 μg, about 1400 μg, about 1500 μg, about 1600 μg, about 1700 μg, about 1800 μg, about 1900 μg, about 2000 μg, about 2100 μg, about 2200 μg, about 2300 μg, about 2400 μg, about 2500 μg, about 2600 μg, about 2700 μg, about 2800 μg, about 2900 μg, or about 3000 μg, including all ranges and values therebetween. In some embodiments, the subject is administered about 100 μg to about 2,000 μg of BT compound per dose. In some embodiments, the subject is administered about 100 μg to about 3,000 μg of BT compound per dose. In some embodiments, the subject is administered about 1000 μg to about 3,000 μg of BT compound per dose. In some embodiments, the subject is administered about 1000 μg to about 2,000 μg of BT compound per dose. In some embodiments, the subject is administered about 2000 μg to about 3,000 μg of BT compound per dose.

In some embodiments, the concentration of bismuth in the lungs after a single daily dose is from about 0.03 μg/g lung tissue to about 3 μg/g lung tissue, e.g. about 0.03 μg/g lung tissue, about 0.1 μg/g lung tissue, about 0.25 μg/g lung tissue, about 0.5 μg/g lung tissue, about 0.75 μg/g lung tissue, about 1 μg/g lung tissue, about 1.25 μg/g lung tissue, about 1.5 μg/g lung tissue, about 1.75 μg/g lung tissue, about 2 μg/g lung tissue, about 2.25 μg/g lung tissue, about 2.5 μg/g lung tissue, about 2.75 μg/g lung tissue, or about 3 μg/g lung tissue, including all ranges and values therebetween. In some embodiments, the concentration of bismuth in the lungs after a single daily dose is from about 0.1 μg/g lung tissue to about 3 μg/g lung tissue. In some embodiments, the concentration of bismuth in the lungs after a single daily dose is from about 1 μg/g lung tissue to about 3 μg/g lung tissue. In some embodiments, the concentration of bismuth in the lungs after 28 daily doses is from about 0.3 μg/g lung tissue to about 60 μg/g lung tissue.

In some embodiments, the BT composition is administered to the subject in need thereof three times per day, two times per day, once daily, every other day, once every three days, once every week, once every other week, once monthly, or once every other month. In some embodiments, the BT composition is administered once per month, twice per month, three times per month, four times per month, once every two weeks, once per week, twice per week, or three times per week. In some embodiments, the BT composition is administered once per month, twice per month, three times per month, or four times per month. In some embodiments, the BT composition is administered once every two weeks, once per week, twice per week, or three times per week. In some embodiments, the BT composition is administered once per week. In some embodiments, the BT composition is administered once daily. In some embodiments, the BT composition is administered once weekly. In some embodiments, the BT composition is administered once monthly. In some embodiments, the BT composition is administered twice monthly. In some embodiments, the BT composition is administered three times monthly. In some embodiments, the BT composition is administered four times monthly. In certain embodiments, the BT composition is administered once every other week. In some embodiments, the BT composition is administered once every three weeks. In some embodiments, the BT composition is administered chronically in a 4 week on/4 week off dosing schedule. In some embodiments, the BT composition is administered chronically, for example as part of a background therapy. In some embodiments, the BT composition is administered as needed.

In some embodiments, the BT composition is administered for a period of less than 24 months, less than 18 months, less than 12 months, less than 9 months, less than 6 months, less than 3 months, or less than 1 month. In some embodiments, the BT composition is administered for a period between 1 month and 24 months. In some embodiments, the BT composition is administered for a period between 1 month and 18 months. In some embodiments, the BT composition is administered for a period between 1 month and 12 months. In some embodiments, the BT composition is administered for a period between 1 month and 6 months. In some embodiments, the BT composition is administered for a period between 1 month and 3 months. In some embodiments, the BT composition is administered for a period between 3 months and 24 months. In some embodiments, the BT composition is administered for a period between 3 months and 18 months. In some embodiments, the BT composition is administered for a period between 3 months and 12 months. In some embodiments, the BT composition is administered for a period between 3 months and 6 months. In some embodiments, the BT composition is administered for a period of 1 to 56 days. In some embodiments, the BT composition is administered for a period of 14 to 28 days. In some embodiments, the BT composition is administered once per month over the treatment period. In some embodiments, the BT composition is administered once every two weeks over the treatment period. In some embodiments, the BT composition is administered once per week over the treatment period. In some embodiments, the BT composition is administered twice per week over the treatment period. In some embodiments, the BT composition is administered three times per week over the treatment period.

In some embodiments, the present disclosure provides a method of reducing NTM intracellular bacterial burden in a subject, comprising contacting an infected cell of the subject with an effective amount of a bismuth-thiol (BT) composition that comprises a BT compound.

In some embodiments, contacting the cell with the BT composition reduces the intracellular bacterial burden by about 10-fold to about 1000-fold. In some embodiments, the intracellular bacterial burden is reduced by about 10-fold, about 50-fold, about 100-fold, about 250-fold, about 500-fold, or about 1000-fold. In some embodiments, contacting the cell with the BT composition keeps the intracellular bacterial burden from increasing, i.e., the BT composition provides a bacteriostatic effect. In some embodiments, contacting the cell with the BT composition results in intracellular accumulation of the BT compound. In some embodiments, contacting the cell with the BT composition results in phagocytosis of the BT compound.

In some embodiments, the infected cell is a phagocyte. In some embodiments, the infected cell is a macrophage cell. In some embodiments, the macrophage cells are THP-1 cells. In some embodiments, the macrophage cells are alveolar macrophages. In some embodiments, the macrophage cells are M1 or M1-like macrophages. In some embodiments, the macrophage cells are infected with one or more strains of M. abscessus and/or M. avium. In some embodiments, the macrophages are infected with one or more strains of M. abscessus and/or M. avium complex. In some embodiments, the macrophage cells are histiocytes. In some embodiments, the macrophage cells are osteoclasts.

In some embodiments, the present disclosure provides a method for treating or providing prophylaxis against a nontuberculous Mycobacterium (NTM) lung infection in a subject in need of treatment or prophylaxis, comprising: administering to the lungs of the subject for an administration period, a BT composition of the present disclosure comprising a BT compound described herein.

In some embodiments, administering to the lungs of the patient comprises aerosolizing the BT composition to provide an aerosolized BT composition, and administering the aerosolized BT composition to the lungs of the subject. In some embodiments, administering the aerosolized BT composition to the lungs of the subject is by a nebulizer, dry powder inhalation, nanoparticle inhalation, metered-dose inhalation, or any other drug inhalation method known in the art. In some embodiments, the dry powder is micronized. In some embodiments, the nanoparticles are lipid nanoparticles. In some embodiments, the nanoparticles are dry nanoparticles. In some embodiments, administering the aerosolized BT composition to the lungs of the subject is by a nebulizer.

In some embodiments, an aerosolized BT composition is administered once per day in a single dosing session during the administration period. In some embodiments, an aerosolized BT composition is administered three times per week in a single dosing session during the administration period. In some embodiments, an aerosolized BT composition is administered twice per week in a single dosing session during the administration period. In some embodiments, an aerosolized BT composition is administered once per week in a single dosing session during the administration period. In some embodiments, an aerosolized BT composition is administered once every two weeks in a single dosing session during the administration period.

In some embodiments, during a single dosing session, the aerosolized BT composition is administered in less than about 75 minutes, less than about 60 minutes, less than about 30 minutes, less than about 15 minutes, or less than about 5 minutes. In some embodiments, during the single dosing session, the aerosolized BT composition is administered in about 60 to about 75 minutes, about 45 minutes to about 60 minutes, about 30 minutes to about 45 minutes, about 20 minutes to about 30 minutes, or about 15 minutes to about 20 minutes.

In some embodiments, an aerosolized BT composition is administered over a 1 week, 2 week, 3 week, 4 week, 1 month, 2 month, 3 month, 4 month, 5 month, 6 month, 12 month, 18 month, or 24 month treatment period. In some embodiments, an aerosolized BT composition is administered over a 1 week, 2 week, 3 week, 4 week, 1 month, 2 month, 3 month, 4 month, 5 month, or 6 month treatment period.

In some embodiments, the present disclosure provides a method for treating a biofilm-associated nontuberculous Mycobacterium (NTM) infection in the lungs of a subject in need, comprising administering to the subject a BT composition of the present disclosure that comprises a BT compound described herein.

In some embodiments, the present disclosure provides a method of treating an NTM infection in a subject having a macrophage infection, comprising administering to the subject an effective amount of a bismuth-thiol (BT) composition of the present disclosure that comprises a BT compound described herein.

In some embodiments, the present disclosure provides a method of treating an NTM infection in a subject, the method comprising: (i) testing for the presence of bacteria-infected macrophages in a biological sample from the subject; and (ii) administering an effective amount of the bismuth-thiol (BT) composition of the present disclosure that comprises a BT compound described herein to the subject if the sample tests positive for bacteria-infected macrophages.

In some embodiments, the macrophages are tested for the presence of one or more pathogenic species of NTM. In some embodiments, the macrophages are tested for the presence of M. abscessus and/or M. avium. In some embodiments, the macrophages are tested for the presence of M. abscessus. In some embodiments, the macrophages are tested for the presence of M. avium.

In some embodiments, the methods disclosed herein further comprise administering an effective amount of an additional antibacterial agent. In some embodiments, the additional antibacterial agent is amikacin, clarithromycin, azithromycin, ethambutol, rifampicin, tigecycline, linezolid, imipenem, cefoxitin, or combination thereof. In some embodiments, the additional antibacterial agent is amikacin or clarithromycin. In some embodiments, the additional antibacterial agent is amikacin. In some embodiments, administration of a BT composition and an additional antibacterial agent results in a synergistic effect in treating the NTM infection. In some embodiments, administration of a BT composition and amikacin results in a synergistic effect in treating the NTM infection. Accordingly, in some embodiments, an effective amount of an additional antibacterial agent is an amount that is ineffective in treating the NTM infection when administered without a BT composition and an effective amount of BT composition is an amount that is ineffective in treating the NTM infection when administered without an additional antibacterial agent.

In some embodiments, the BT composition and the additional antibacterial agent are administered simultaneously, separately, or sequentially. In some embodiments, the BT composition is administered concurrently with, prior to, or after the additional antibacterial agent.

In some embodiments, the BT composition and the additional antibacterial agent are in combination to a subject in need thereof. In some embodiments, the BT composition and the additional antibacterial agent are coadministered to a subject in need thereof. In some embodiments, the BT composition and the additional antibacterial agent are conjointly administered to a subject in need thereof.

### Bismuth-Thiol Compositions

In some embodiments, the BT composition comprises a BT compound. In some embodiments, the BT compound is selected from the group consisting of BisBAL, BisEDT, Bis-dimercaprol, BisDTT, Bis-2-mercaptoethanol, Bis-DTE, Bis-Pyr, Bis-Ery, Bis-Tol, Bis-BDT, Bis-PDT, Bis-Pyr/Bal, Bis-Pyr/BDT, BisPyr/EDT, Bis-Pyr/PDT, Bis-Pyr/Tol, Bis-Pyr/Ery, bismuth-1-mercapto-2-propanol, BisEDT/CSTMN (1:1), BisPYR/CSTMN (1:1), BisBAL/CSTMN (1:1), BisTOL/CSTMN (1:1), and BisEDT/2-hydroxy-1-propanethiol.

In some embodiments, the BT composition comprises a BT compound selected from the group consisting of:

bismuth-2,3-dimercaptopropanol (2:3 molar ratio, BisBAL)
bismuth-dithioerythritol (2:3 molar ratio, BisERY)
bismuth-4-methyl-1,2-benzenedithiol (2:3 molar ratio, BisTOL)
bismuth-2,3-butanedithiol (BisBDT)
bismuth-2,3-butanedithiol, 2-mercaptopyridine N-oxide (2:1:2 molar ratio, BisBDT/PYR)
bismuth-2,3-dimercaptopropanol, 2-mercaptopyridine N-oxide (2:1:2 molar ratio,
BisBAL/PYR)
bismuth-1,2-ethanedithiol, 2-mercaptopyridine N-oxide (2:1:2 molar ratio, BisEDT/PYR)
bismuth-4-methyl-1,2-benzenedithiol, 2-mercaptopyridine N-oxide (2:1:2 molar ratio,
BisTOL/PYR)
bismuth-1,3-propanedithiol, 2-mercaptopyridine N-oxide (2:1:2 molar ratio, BisPDT/PYR)
bismuth-dithioerythritol, 2-mercaptopyridine N-oxide (2:1:2 molar ratio, BisERY/PYR)
bismuth-1-mercapto-2-propanol, 1,2-ethanedithiol (1:1:1 molar ratio, BisHPT/EDT)
bismuth with ethanedithiol and 2-mercaptobenzoimidazole (BisEDT/2MBI (1:1))
bismuth with ethanedithiol and 2-mercaptopyrimidine (BisEDT/SPN (2MPMD) (1:1))
bismuth with ethanedithiol and 3-mercapto-1,2,4-triazole (BisEDT/3MTZ (1:1))
bismuth with ethanedithiol and 1-propane thiol (BisEDT/PT (1:1))
bismuth with ethanedithiol and cysteamine (BisEDT/CSTMN (1:1))
bismuth with ethanedithiol and 3-mercaptopropionic acid (BisEDT/3MPA (1:1))
bismuth with lipoic acid (reduced) (BisALA (BisLipo) (1:1.5))
bismuth with 2-mercaptolpyridine N-oxide and 2-mercaptobenzoimidazole (BisPYR/2MBI
(1:1))
bismuth with 2-mercaptolpyridine N-oxide and cysteamine (BisPYR/CSTMN (1:1))
bismuth with 2,3-dimercapto-1-propanol and 2-mercaptobenzoimidazole (BisBAL/2MBI
(1:1))
bismuth with 2,3-dimercapto-1-propanol and cysteamine (BisBAL/CSTMN (1:1))
bismuth with 3,4 dimercapto toluene and 2-mercaptobenzoimidazole (BisTOL/2MBI (1:1))
bismuth with 3,4 dimercapto toluene and cysteamine (BisTOL/CSTMN (1:1))
bismuth with 2-mercapto pyridine (BisEDT/MPYR)

In some embodiments, the BT compound is selected from the group consisting of BisEDT, Bis-Bal, Bis-Pyr, Bis-Ery, Bis-Tol, Bis-BDT, or BisEDT/2-hydroxy-1-propane thiol.

In some embodiments, the BT compound is BisEDT or BisBAL. In some embodiments, the BT compound is BisEDT. In some embodiments, the BisEDT is a compound having the structure:

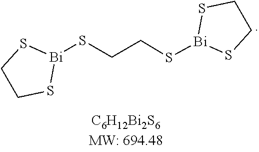

In some embodiments, the BT compound of the present disclosure exhibits a bactericidal effect. In some embodiments, the BT compound exhibits a bacteriostatic effect.

When administered to subject, such as a human, the composition or the compound is preferably administered as a pharmaceutical composition comprising, for example, a compound of the disclosure and a pharmaceutically acceptable carrier. Pharmaceutically acceptable carriers are well known in the art and include, for example, aqueous solutions such as water, physiologically buffered saline, physiologically buffered phosphate, or other solvents or vehicles such as glycols, glycerol, oils such as olive oil, or injectable organic esters. In some embodiments, when such pharmaceutical compositions are for human administration, the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as lyophile for reconstitution, powder, solution, syrup, injection or the like. The composition can also be present in a solution suitable for topical administration.

A pharmaceutically acceptable carrier can contain physiologically acceptable agents that act, for example, to stabilize, increase solubility or to increase the absorption of a compound such as a compound of the disclosure. Such physiologically acceptable agents include, for example, carbohydrates, such as glucose, sucrose, or dextrans; antioxidants, such as ascorbic acid or glutathione; chelating agents; low molecular weight proteins; salts; or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the disclosure. Liposomes, for example, which comprise phospholipids or other lipids, are nontoxic, physiologically acceptable and metabolizable carriers that are relatively simple to make and administer.

Other examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, methyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols and sugar alcohols, such as glycerin, sorbitol, mannitol, xylitol, erythritol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances, including salts such as sodium chloride, employed in pharmaceutical formulations.

The formulations can conveniently be presented in unit dosage form and can be prepared by any methods well known in the art of pharmacy. The amount of active ingredient which can be combined with a carrier material to produce a single dosage form will vary depending upon the subject being treated, the particular mode of administration. The amount of active ingredient that can be combined with a carrier material to produce a single dosage form will generally be that amount of the compound which produces a therapeutic effect.

In some embodiments, the BT composition further comprises one or more carriers selected from animal and vegetable fats, oils, waxes, paraffins, starch, tragacanth, cellulose derivatives, polyethylene glycols, silicones, bentonites, silicic acid, polymers, talc, and zinc oxide. In some embodiments, the carrier is methylcellulose. In some embodiments, the carrier is poly(methyl methacrylate).

Compositions can also be formulated so as to provide slow or controlled release of the active ingredient therein using, for example, hydroxypropyl methylcellulose (HPMC) in varying proportions to provide the desired release profile, other polymer matrices, liposomes and/or microspheres. They can be sterilized by, for example, filtration through a bacteria-retaining filter, by ionizing radiation (gamma photons for example), autoclaving, or by incorporating sterilizing agents in the form of sterile solid compositions that can be dissolved in sterile water, or some other sterile injectable medium immediately before use.

Suspensions, in addition to the active compounds, can contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound can be mixed under sterile conditions with a pharmaceutically acceptable carrier, and with any preservatives or buffers that can be required.

The BT compositions, in addition to an active compound, one or more excipients or carriers, such as animal and vegetable fats, oils, waxes, paraffins, starch, tragacanth, cellulose derivatives, polyethylene glycols, silicones, bentonites, silicic acid, talc, polymers, salts, and zinc oxide, or mixtures thereof. In some embodiments, the BT composition is in the form of an aqueous solution. In some embodiments, the excipient comprises a salt selected from sodium chloride or potassium chloride. In some embodiments, the excipient comprises sodium chloride.

In some embodiments, the BT composition is a powder, spray, ointment, paste, cream, lotion, solution, patch, suspension or gel. In some embodiments, the BT composition is a solution. In some embodiments, the BT composition is an aerosol.

The BT composition can comprise any suitable concentration of bismuth-thiol compound. In some embodiments, the BT composition is administered as a dosage from about 0.25 mg/mL to about 15 mg/mL, from about 0.4 mg/mL to about 15 mg/mL, from about 0.6 mg/mL to about 15 mg/mL, from about 0.6 mg/mL to about 100 mg/mL, from about 5 mg/mL to about 100 mg/mL, from about 10 mg/mL to about 100 mg/mL, from about 25 mg/mL to about 100 mg/mL, from about 50 mg/mL to about 100 mg/mL, from about 0.8 mg/mL to about 15 mg/mL, from about 1 mg/mL to about 10 mg/mL, from 2.5 mg/mL to about 10 mg/mL, from about 4 mg/mL to about 10 mg/mL, from about 5 mg/mL to about 10 mg/mL, from about 6 mg/mL to about 10 mg/mL, 0.6 mg/mL to about 6 mg/mL, from about 4 mg/mL to about 15 mg/mL, from about 6 mg/mL to about 15 mg/mL, from about 50 μg/mL to about 750 μg/mL, from about 75 μg/mL to about 500 μg/mL, from about 100 μg/mL to about 250 μg/mL, from about 100 μg/mL to about 150 μg/mL, or from about 75 μg/mL to about 150 μg/mL; and/or the total amount of the BT composition administered to the lungs is from about 0.25 mg to about 15 mg, from about 0.4 mg to about 15 mg, from about 0.6 mg to about 15 mg, from about 0.8 mg to about 15 mg, from about 1 mg to about 10 mg, from 2.5 mg to about 10 mg, from about 4 mg to about 10 mg, from about 5 mg to about 10 mg, from about 6 mg to about 10 mg, 0.6 mg to about 6 mg, from about 4 mg to about 15 mg, from about 6 mg to about 15 mg, from about 50 μg to about 750 μg, from about 75 μg to about 500 μg, from about 100 μg to about 250 μg, from about 100 μg to about 150 μg, or from about 75 μg to about 150 μg. In certain embodiments, the BT composition is administered as a dosage from about 0.6 mg/mL to about 6 mg/mL.

In some embodiments, the concentration of bismuth in the lungs after a single daily dose is from about 0.03 μg/g lung tissue to about 3 μg/g lung tissue, e.g. about 0.03 μg/g lung tissue, about 0.1 μg/g lung tissue, about 0.25 μg/g lung tissue, about 0.5 μg/g lung tissue, about 0.75 μg/g lung tissue, about 1 μg/g lung tissue, about 1.25 μg/g lung tissue, about 1.5 μg/g lung tissue, about 1.75 μg/g lung tissue, about 2 μg/g lung tissue, about 2.25 μg/g lung tissue, about 2.5 μg/g lung tissue, about 2.75 μg/g lung tissue, or about 3 μg/g lung tissue, including all ranges and values therebetween. In some embodiments, the concentration of bismuth in the lungs after a single daily dose is from about 0.1 μg/g lung tissue to about 3 μg/g lung tissue. In some embodiments, the concentration of bismuth in the lungs after a single daily dose is from about 1 μg/g lung tissue to about 3 μg/g lung tissue. In some embodiments, the concentration of bismuth in the lungs after 28 daily doses is from about 0.3 μg/g lung tissue to about 60 μg/g lung tissue.

In some embodiments, the BT composition is administered three times per day, two times per day, once daily, every other day, once every three days, once every week, once every other week, once monthly, or once every other month. In some embodiments, the BT composition is administered once per month, twice per month, three times per month, four times per month, once every two weeks, once per week, twice per week, or three times per week. In some embodiments, the BT composition is administered once per month, twice per month, three times per month, or four times per month. In some embodiments, the BT composition is administered once every two weeks, once per week, twice per week, or three times per week. In some embodiments, the BT composition is administered once per week. In some embodiments, the BT composition is administered once daily. In certain embodiments, the BT composition is administered once weekly. In certain embodiments, the BT composition is administered once every other week. In some embodiments, the BT composition is administered chronically in a 4 week on/4 week off dosing schedule. In some embodiments, the BT composition is administered chronically, for example as part of a background therapy. In some embodiments, the BT composition is administered as needed. As will be appreciated by a person having ordinary skill in the art, the administration frequency may depend on a number of factors including dose and administration route. For example, if the BT composition is administered via an aerosol administration, a low dose such as 100-1000 μg/mL may be administered once or twice daily; however, a high dose such as 2.5-10 mg/mL may be administered e.g., once or twice a week.

In some embodiments of the present disclosure, the BT composition is a suspension of a BT compound in polysorbate (e.g. polysorbate 80) and/or in a buffer (e.g. sodium phosphate buffer). For example, in some embodiments, the BT composition is a suspension of the BT compound in from about 0.1% polysorbate 80 to about 1.0% polysorbate 80, including all ranges therebetween. For example, the BT composition is a suspension of the BT compound in about 0.1% polysorbate 80, about 0.2% polysorbate 80, about 0.3% polysorbate 80, about 0.4% polysorbate 80, about 0.5% polysorbate 80, about 0.6% polysorbate 80, about 0.7% polysorbate 80, about 0.8% polysorbate 80, about 0.9% polysorbate 80, or about 1% polysorbate 80. In some embodiments, the BT composition is a suspension of the BT compound in about 0.5% polysorbate 80.

In some embodiments, the present disclosure provides a bismuth-thiol (BT) composition that comprises a BT compound (e.g., BisEDT)) suspended therein, wherein the BT composition comprises a plurality of particles. In some embodiments, the present disclosure provides a bismuth-thiol (BT) composition that comprises a BT compound (e.g., BisEDT)) suspended therein, wherein the BT composition comprises a plurality of microparticles. In some embodiments, the D90 of said particles/microparticles is less than or equal to 4.5 μm, or 4.0 μm, or 3.5 μm, or 3.0 μm, or 2.5 μm, or 2.0 μm, or 1.9 μm, or 1.8 μm, or m 1.7 μm, or 1.6 μm, or 1.5 μm or any ranges in between. In some embodiments, the D90 of said particles/microparticles is less than or equal to 1.9 μm. In another embodiment, the D90 of said particles/microparticles is less than or equal to 1.6 μm. In another embodiment, the D50 of said particles/microparticles is less than or equal to 2.5 μm, or 2.0 μm, or 1.5 μm, or 1.3 μm, or 1.2 μm, or 1.1 μm, or 1.0 μm, or 0.9 μm, or 0.87 μm, or 0.72 μm or any ranges in between. In another embodiment, the D10 of said particles/microparticles is less than or equal to 0.9 μm, or 0.8 μm, or 0.7 μm, or 0.6 μm, or 0.50 μm, or 0.40 μm, or 0.39 μm, or 0.38 μm, or 0.37 μm, or 0.36 μm, or 0.35 μm, or 0.34 μm, or 0.33 μm, or any ranges in between.

In some embodiments, the bismuth-thiol (BT) composition of the present disclosure comprises a BT compound suspended therein, wherein the BT composition comprises a plurality of particles, wherein the D90 of said particles is less than or equal to about 1.6 μm. In some embodiments, the bismuth-thiol (BT) composition comprises a BT compound (e.g., BisEDT) suspended therein, wherein the BT composition comprises a plurality of microparticles, wherein the D90 of said microparticles is less than or equal to about 1.6 μm.

In some embodiments, the BT composition comprises a BT compound (e.g., BisEDT) at a concentration greater than about 0.1 mg/mL, about 0.05% to about 1.0% polysorbate 80 (Tween 80@), about 0.05 to 40 mM sodium chloride, and optionally about 2 to 20 mM sodium phosphate at about pH 7.4.

A variety of buffers may be used in the context of the present disclosure and will be readily apparent to a person having ordinary skill in the art. For example, in some embodiments, suitable buffers include sodium or potassium citrate, citric acid, phosphate buffers such as sodium phosphate, boric acid, sodium bicarbonate and various mixed phosphate buffers including combinations of Na₂HPO₄, NaH₂PO₄ and KH₂PO₄. In some embodiments, sodium phosphate buffer is used. In some embodiments, sodium citrate buffer is used. Without being bound by any particular theory, changes in airway surface liquid pH may contribute to the host defense defect in cystic fibrosis soon after birth. Changes in lung pH may impact the airway surface liquid environment, improve airway defenses, and alter the disease course. Accordingly, the formulation pH may vary from about 5 to about 10. In some embodiments, the formulation pH is about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or about 10. In some embodiments, the formulation pH is about 7.4.

In some embodiments, the BT composition is a suspension of a BT compound in about 0.5% polysorbate 80 in sodium phosphate buffer at a pH of about 7.4. In some embodiments, the BT compound is present in the composition at a concentration ranging from about 100 μg/mL to about 1000 mg/mL including all integers and ranges therebetween. For example, in some embodiments, the BT compound is present in the composition at a concentration ranging from about 100 μg/mL, 200 μg/mL, 300 μg/mL, 400 μg/mL, 500 μg/mL, 600 μg/mL, 700 μg/mL, 800 μg/mL, 900 μg/mL, 1000 μg/mL, 10 mg/mL, 25 mg/mL, 50 mg/mL, 100 mg/mL, 125 mg/mL, 150 mg/mL, 175 mg/mL, 200 mg/mL, 225 mg/mL, 250 mg/mL, 275 mg/mL, 300 mg/mL, 325 mg/mL, 350 mg/mL, 375 mg/mL, 400 mg/mL, 425 mg/mL, 450 mg/mL, 475 mg/mL, 500 mg/mL, 525 mg/mL, 550 mg/mL, 575 mg/mL, 600 mg/mL, 625 mg/mL, 650 mg/mL, 675 mg/mL, 700 mg/mL, 725 mg/mL, 750 mg/mL, 775 mg/mL, 800 mg/mL, 825 mg/mL, 850 mg/mL, 875 mg/mL, 900 mg/mL, 925 mg/mL, 950 mg/mL, 975 mg/mL, to about 1000 mg/mL. In some embodiments, the BT compound is present in the composition at a concentration ranging from about 100 μg/mL to about 1000 μg/mL.

In some embodiments, the composition osmolality is further adjusted with an additive such as NaCl or TDAPS to achieve a desired osmolality. For example, in some embodiments, the osmolality of the composition is adjusted with sodium chloride to an osmolality ranging from about 100 mOsmol/kg to about 500 mOsmol/kg, including all integers and ranges therebetween. In some embodiments, the osmolality of the composition is from about 290 mOsmol/kg to about 310 mOsmol/kg. For example, in some embodiments, the osmolality of the composition is about 290 mOsmol/kg, 291 mOsmol/kg, 292 mOsmol/kg, 293 mOsmol/kg, 294 mOsmol/kg, 295 mOsmol/kg, 296 mOsmol/kg, 297 mOsmol/kg, 298 mOsmol/kg, 299 mOsmol/kg, 300 mOsmol/kg, 301 mOsmol/kg, 302 mOsmol/kg, 303 mOsmol/kg, 304 mOsmol/kg, 305 mOsmol/kg, 306 mOsmol/kg, 307 mOsmol/kg, 308 mOsmol/kg, 309 mOsmol/kg, to about 310 mOsmol/kg. In some embodiments, the osmolality is about 300 mOsmol/kg.

In some embodiments, the BT composition is a suspension of BisEDT in polysorbate (e.g. polysorbate 80) in a buffer (e.g. sodium phosphate buffer). In some embodiments, the BT composition is a suspension of BisEDT in about 0.5% polysorbate 80 in a sodium phosphate buffer at a pH of about 7.4. In some embodiments, the BT composition is a suspension of BisEDT in about 0.5% polysorbate 80 in a sodium phosphate buffer at a pH of about 7.4, wherein the composition has an osmolality of about 300 mOsmol/kg (e.g. adjusted to 300 mOsmol/kg with sodium chloride). In some embodiments, the BisEDT is present at a concentration of about 100 μg/mL, 250 μg/mL, 500 μg/mL, 750 μg/mL, 1000 μg/mL, 2.5 mg/mL, 10 mg/mL, 25 mg/mL, 50 mg/mL, 75 mg/mL, or about 100 mg/mL.

In some embodiments, the BT composition is delivered to the lungs of a subject. In some embodiments, the BT composition is administered to the lungs of a subject by a nebulizer, dry powder inhalation, nanoparticle inhalation, metered-dose inhalation, or any other drug inhalation method known in the art. In some embodiments, the dry powder is micronized. In some embodiments, the nanoparticles are lipid nanoparticles. In some embodiments, the nanoparticles are not lipid nanoparticles. In some embodiments, the nanoparticles are dry nanoparticles. In some embodiments, the BT composition is a suspension formulation which is intended for pulmonary delivery. For example, the BT composition is a suspension formulation which is ultimately administered by inhalation either orally and/or nasally.

Accordingly, in some embodiments, the BT composition is in the form of an aerosol. In some embodiments, the BT composition is aerosolized by a device such as a nebulizer. In some embodiments, the aerosol comprises a plurality of dispersed liquid droplets in a gas, said liquid droplets comprising a BT composition comprising bismuth-1,2-ethanedithiol (BisEDT) suspended therein, wherein the BT composition comprises a plurality of BisEDT particles/microparticles having a D90 as disclosed herein, e.g., a D90 of less than about 2 μm; and wherein at least 70%, at least 80%, or at least 90% of the liquid droplets have a MMAD as disclosed herein, e.g., a MMAD from about 0.4 μm to about 5 μm as measured by cascade impaction or laser time of flight.

In some embodiments, the aerosol is administered by inhalation, orally or nasally, using an aerosol device, such as a nebulizer. Known nebulizers, such as PARI IC Plus, can administer the disclosed aerosols as an aqueous solution, optionally in buffered saline. The solution can be provided to the subject in the form of an ampule for use in the nebulizer. The nebulizer can be reusable and includes a compressor that provides the formulation over a period of time, such as about 10-15 minutes or longer, e.g., for a period of 45-60 minutes. Known compressors, such as APRI Vios Air and DeVilbiss Pulmo-aide, are suitable for administration. The nebulizer administers the formulation topically to the lung tissues, such as mucosa, the bronchi and/or the bronchioles, alveoli, deep lung alveoli. The formulation can penetrate lung mucosa and biofilms to reduce the microbial (e.g. bacterial or fungal) biofilm, impair the growth of the microbial (e.g. bacterial or fungal) biofilm, prevent reformation of the microbial (e.g. bacterial or fungal) biofilm, reduce planktonic growth, and/or inhibit planktonic growth.

In other embodiments, a nose-only aerosol device can be used for administration of the formulation.

Powders and sprays can contain, in addition to an active compound, excipients such as methylcellulose, sodium chloride, PMMA, lactose, talc, silicic acid, aluminum hydroxide, calcium silicates and polyamide powder, dipalmitoylphosphatidylcholine (DPPC), leucine, polyethyleneglycol, or mixtures of these substances. Sprays can additionally contain customary propellants, such as chlorofluorohydrocarbons and volatile unsubstituted hydrocarbons, such as butane and propane.

An exemplary BT composition formulation is a neutral pH, isotonic, buffered aqueous solution of BT compound particles/microparticles with a nonionic surfactant. In certain embodiments, the buffer is a phosphate buffer with added NaCl. In some embodiments, the microparticle size is a D₅₀ of about 1-5 μm. The formulation can be delivered using commercially available compressed air jet nebulizer. In some embodiments, the formulation concentration is about 0.1 μg/mL to about 100 mg/mL.

In some embodiments, the present disclosure provides an aerosol composition comprising a plurality of dispersed liquid droplets in a gas, said liquid droplets comprising a BT compound suspended therein, wherein at least 60%, 65%, 70%, 75%, 80%, 90%, or 95% of the liquid droplets have a mass median aerodynamic diameter (MMAD) from about 0.4 μm to about 5 μm when measured by laser time of flight and/or cascade impactor. In some embodiments, at least 60%, 65%, 70%, 75%, 80%, 90%, or 95% of the liquid droplets have a MMAD of from about 0.4 μm to about 7 μm, or from about 0.5 μm to about 5 μm, or from about 0.7 μm to about 4 μm, or from about 0.7 μm to about 3.5 μm, or from about 0.8 μm to about 3.5 μm, or from about 0.9 μm to about 3.5 μm, or from about 0.9 μm to about 3 μm, or from about 0.8 μm to about 1.8 μm, or from about 0.8 μm to about 1.6 μm, or from about 0.9 μm to about 1.4 μm, or from about 1.0 μm to about 2.0 μm, or from about 1.0 μm to about 1.8 μm, including all ranges therebetween. In some embodiments, at least 60%, 65%, 70%, 75%, 80%, 90%, or 95% of the liquid droplets have a MMAD of from about 0.8 μm to about 1.6 μm, or from about 0.9 μm to about 3.5 μm, or from about 0.9 μm to about 3 μm, or from about 0.9 μm to about 1.4 μm, or from about 1.0 μm to about 2.0 μm, or from about 1.0 μm to about 1.8 μm, and all ranges therebetween.

In some embodiments, the plurality of liquid droplets have a D90 of less than about 10 μm. For example, in some embodiments, the plurality of liquid droplets have a D90 of less than about 10 μm, 9 μm, 8 μm, 7 μm, 6 μm, 5 μm, 4 μm, 3 μm, 2 μm, or about 1 μm. In some embodiments, the plurality of liquid droplets have a D90 of less than about 3 μm. In some embodiments, the plurality of liquid droplets have a D90 ranging from about 1 μm to about 5 μm, or about 2 μm to about 6 μm, or about 2 μm to about 4 μm, or about 2 μm to about 3 μm, or about 1 μm to about 4 μm, or about 1 μm to about 3 μm.

In some embodiments, the plurality of liquid droplets are dispersed in a continuous gas phase.

In some embodiments, the BT compound (e.g. BisEDT) is suspended in the liquid droplet. The BT compounds of the present disclosure may have little to no solubility in conventional solvents and aerosol carriers, and therefore exist substantially as a suspension of BT particles in the aerosol droplet. For example, in some embodiments, the BT compound (such as BisEDT) is less than 1% soluble in the aerosol carrier and therefore exists primarily (>99%) as a solid.

In some embodiments, the droplets further comprise polysorbate 80 (e.g. from about 0.05% to about 1%) and optionally a buffer (e.g. sodium phosphate or sodium citrate) at a pH of about 7.4; and/or sodium chloride.

The aerosols of the present disclosure have a very narrow MMAD distribution, which is beneficial because of the need to concentrate the particle mass in the target size range, and minimize or eliminate the fraction of the product that is outside of the respirable range or ‘fines’, i.e. particles of typically less than 0.4 μm diameter. The ability to create a narrow droplet size distribution in the appropriate size range provides control of the initial evaporation rate and allows for high deposition efficiency. The limiting factor in terms of the lower limit of particle aerosol droplet size is the BT particle size (e.g. the BisEDT particle size). An aerosolized droplet cannot be smaller than the BisEDT particulate size. As such, the BT particle size distribution, as well as the uniformity and consistent reproducibility of the BT particulate size distribution, are important beneficial characteristics to support the generation of a safe, effective, and efficient aerosolized BisEDT drug product for inhalation purposes. Accordingly, in some embodiments, the aerosols of the present disclosure effectuate a deposition efficiency of greater than 3%, greater than 5%, greater than 10%, greater than 15%, greater than 20%, greater than 25%, greater than 30%, greater than 35% m greater than 40%, greater than 45%, greater than 50%, greater than 55%, greater than 60%, greater than 65%, greater than 70%, greater than 75%, and greater than 80%. In some embodiments, the deposition efficiency refers to deposition to the deep lung region of lung, for example, to the deep lung alveoli. In some embodiments, the aerosols of the present disclosure effectuate a deposition efficiency upon aerosolization via a nebulizer. For example, the nebulizer is a jet nebulizer. In some embodiments, the jet nebulizer is a Pari LC Plus jet nebulizer or Pari LC SPRINT jet nebulizer. In some embodiments, the nebulizer has an inlet pressure from about 10 to about 40 psig (e.g. 20-25 psig). In some embodiments, the inlet flow is from about 3 L/min to about 8 L/min (e.g. 5.2 L/min). In some embodiments, the exhaust air flow is from about 3 L/min to about 8 L/min (e.g. 5 L/min).

The alveolar region of the lung has a minimal thickness (0.5 μm-2.5 μm) separating the blood flow from the lumen so conventional pulmonary agents that deposit on the alveolar epithelium have extremely short lung residence time due to systemic absorption. Accordingly, conventional pulmonary treatments typically require frequent dosing in order to maintain adequate levels of drug at the tissue level. However, the aerosolized particles of the present disclosure were surprisingly discovered to possess an exceptionally long residence time in the lungs (measured as half-life) and have reduced mucociliary clearance and macrophage uptake relative to conventional pulmonary treatments. Furthermore, the long residence time of the aerosols of the present minimizes systemic activity and associated systemic side effects. Without being bound by any particular theory, it is believed that the aerosolized particles/microparticles dissolve slowly on the lung lumen and the systemic exposure is thus dissolution rate limited. Further, the increased lung residence time results in significant reductions in microbial colony due to the continuous presence of the BT particles/microparticles.

Accordingly, in some embodiments, when the aerosol is deposited to the lung (e.g. to the deep lung alveoli), the BT compounds have an average half-life of at least 2 days. For example, the BT compounds have an average half-life of about 2, 3, 4, or 5 days. In some embodiments, the BT compound is BisEDT. In a specific embodiment, the lung tissue half-life of BisEDT is 30 h or more, 40 h or more, 50 h or more, 60 h or more, 70 h or more, 80 h or more, 90 h or more, 100 h or more, 110 h or more, 125 h or more, or 150 h or more. In a specific embodiment, the lung tissue half-life is after a single dose via inhalation. In another embodiment, lung tissue is from a rat. In another embodiment, lung tissue half-life of BisEDT is determined by the use of protocol as in Example 8 herein.

In another embodiment, the lung tissue half-life of BisEDT is 80 h or more when the rat is given a single dose of 100 μg/kg lung using a Pari LC plus jet nebulizer to administer to the rats with the formulations described herein. In another embodiment, the lung tissue half-life of BisEDT is 90 h or more. In another embodiment, the lung tissue half-life of BisEDT is 100 h or more.

In another embodiment, after delivering the aerosolized composition to a subject, at least 60%, 65%, 70%, 75%, 80%, 90%, or 95% of the dose is deposited on the lung, as opposed to the oropharyngeal region and the conducting airways. In a specific embodiment, at least 80% of the dose is deposited on the lung, as opposed to the oropharyngeal region and the conducting airways. In another embodiment, at least 90% of the dose is deposited on the lung, as opposed to the oropharyngeal region and the conducting airways.

It was previously unheard of for an aerosolized pulmonary treatment to have aerosol particles with a narrow distribution that effectuate a high deposition efficiency coupled with an exceptionally long lung residence time for continuous treatment and little to no systemic absorption.

In some embodiments, after delivering the aerosolized composition to a subject, at least 60%, 65%, 70%, 75%, 80%, 90%, or 95% of the dose is deposited on the lung, as opposed to the oropharyngeal region and the conducting airways. In some embodiments, at least 80% of the dose is deposited on the lung, as opposed to the oropharyngeal region and the conducting airways. In another embodiment, at least 90% of the dose is deposited on the lung, as opposed to the oropharyngeal region and the conducting airways. In another embodiment, the percent deposition is determined using a Pari LC plus jet nebulizer to administer to the rats with the formulations described herein.

In another embodiment, the lung tissue half-life of BisEDT is 80 h or more when the rat is given a single dose of 100 μg/kg lung using a Pari LC plus jet nebulizer to administer to the rats with the formulations described herein. In another embodiment, the lung tissue half-life of BisEDT is 90 h or more. In another embodiment, the lung tissue half-life of BisEDT is 100 h or more.

In another embodiment, the methods of the present invention include treating, managing or lessening the severity of cystic fibrosis (CF) symptoms associated with an NTM infection in a subject, comprising administering to the subject an aerosol comprising a plurality of dispersed liquid droplets, wherein the liquid droplets comprise a bismuth-thiol (BT) composition that comprises particles of a BT compound suspended therein, wherein the particles have a D90 of less than about 5 μm (e.g., as measured by laser diffraction), and/or wherein at least 70%, 80%, or 90% of the liquid droplets have a mass median aerodynamic diameter (MMAD) from about 0.4 μm to about 5 μm (e.g., as measured by cascade impaction or laser time of flight).

In another embodiment, the methods of the present invention include treating, managing or lessening the severity of cystic fibrosis (CF) symptoms associated with an NTM infection in a subject, comprising administering to the subject an aerosol comprising a plurality of dispersed liquid droplets, wherein the liquid droplets comprise a bismuth-thiol (BT) composition that comprises microparticles of a BT compound suspended therein, wherein the microparticles have a D90 of less than about 5 μm (e.g., as measured by laser diffraction), and/or wherein at least 70%, 80%, or 90% of the liquid droplets have a mass median aerodynamic diameter (MMAD) from about 0.4 μm to about 5 μm (e.g., as measured by cascade impaction or laser time of flight).

In some embodiments, the composition is a suspension of particles/microparticles having a volumetric mean diameter (VMD) from about 0.4 μm to about 5 μm. In some embodiments, at least 60%, 65%, 70%, 75%, 80%, 90%, or 95% of the particles/microparticles have a VMD of from about 0.4 μm to about 5 μm, or from about 0.6 μm to about 2.5 μm, or from about 0.7 μm to about 4 μm, or from about 0.7 μm to about 3.5 μm, or from about 0.7 μm to about 3.0 μm, or from about 0.9 μm to about 3.5 μm, or from about 0.9 μm to about 3 μm, or from about 0.8 μm to about 1.8 μm, or from about 0.8 μm to about 1.6 μm, or from about 0.9 μm to about 1.4 μm, or from about 1.0 μm to about 2.0 μm, or from about 1.0 μm to about 1.8 μm and all ranges therebetween. In some embodiments, at least 60%, 65%, 70%, 75%, 80%, 90%, or 95% of the particles/microparticles have a VMD of from about 0.6 μm to about 2.5 μm, or from about 0.8 μm to about 1.6 μm, or from about 0.9 μm to about 3.5 μm, or from about 0.9 μm to about 3 μm, or from about 0.9 μm to about 1.4 μm, or from about 1.0 μm to about 2.0 μm, or from about 1.0 μm to about 1.8 μm and all ranges therebetween. In some embodiments, the particles/microparticles have a D90 of less than 5 μm, 4 μm, 3 μm, 2 μm, or about 1 μm. In some embodiments, the particles/microparticles have a D90 of less than about 3 μm. In some embodiments, the particles/microparticles have a D90 ranging from about 1 μm to about 5 μm, or about 2 μm to about 4 μm, or about 2 μm to about 3 μm, or about 1 μm to about 4 μm, or about 1 μm to about 3 μm, or about 1 μm to about 2 μm.

In some embodiments, the dispersed liquid droplets have a MMAD from about of from about 0.4 μm to about 5 μm. In some embodiments, at least 60%, 65%, 70%, 75%, 80%, 90%, or 95% of the liquid droplets have a MMAD of from about 0.4 μm to about 7 μm, or from about 0.5 m to about 5 μm, or from about 0.7 μm to about 4 μm, or from about 0.7 μm to about 3.5 μm, or from about 0.8 μm to about 3.5 μm, or from about 0.9 μm to about 3.5 μm, or from about 0.9 μm to about 3 μm, or from about 0.8 μm to about 1.8 μm, or from about 0.8 μm to about 1.6 μm, or from about 0.9 μm to about 1.4 μm, or from about 1.0 μm to about 2.0 μm, or from about 1.0 μm to about 1.8 μm and all ranges therebetween. In some embodiments, at least 60%, 65%, 70%, 75%, 80%, 90%, or 95% of the liquid droplets have a MMAD of from about 0.8 μm to about 1.6 m, or from about 0.9 μm to about 3.5 μm, or from about 0.9 μm to about 3 μm, or from about 0.9 m to about 1.4 μm, or from about 1.0 μm to about 2.0 μm, or from about 1.0 μm to about 1.8 μm and all ranges therebetween. In some embodiments, the plurality of liquid droplets have a D90 of less than about 10 μm. For example, in some embodiments, the plurality of liquid droplets have a D90 of less than about 10 μm, 9 μm, 8 μm, 7 μm, 6 μm, 5 μm, 4 μm, 3 μm, 2 μm, or about 1 μm. In some embodiments, the plurality of liquid droplets have a D90 of less than about 3 μm. In some embodiments, the plurality of liquid droplets have a D90 ranging from about 1 μm to about 5 μm, or about 2 μm to about 6 μm, or about 2 μm to about 4 μm, or about 2 μm to about 3 μm, or about 1 μm to about 4 μm, or about 1 μm to about 3 μm.

In some embodiments, the plurality of liquid droplets are dispersed in a continuous gas phase.

In some embodiments, the aerosol comprises a BT compound disclosed herein.

In some embodiments of the presently disclosed compositions, at least 60%, 65%, 70%, 75%, 80%, 90%, or 95% of the particles/microparticles have a volumetric mean diameter of from about 0.6 μm to about 2.5 μm. In some embodiments, substantially all of the particles/microparticles have a VMD of from about 0.6 μm to about 2.5 μm. In some embodiments, at least 70% of the dispersed liquid droplets have a MMAD of about 0.9 μm to about 3 μm. In some embodiments, the composition is a suspension of particles/microparticles having a volumetric mean diameter (VMD) from about 0.6 μm to about 2.5 μm and/or a mass median aerodynamic diameter (MMAD) from about 0.9 μm to about 3 μm. In some embodiments, the bismuth-thiol (BT) composition comprises a plurality of particles/microparticles that comprise a BT compound, substantially all of said particles/microparticles having a volumetric mean diameter of from about 0.4 μm to about 5 μm, wherein the BT compound is BisEDT or BisBAL. In some embodiments, the BT compound is BisEDT.

In some embodiments, the compositions disclosed herein are aerosolized via a nebulizer. For example, the nebulizer is a jet nebulizer or vibrating mesh nebulizer. In some embodiments, the jet nebulizer is a Pari LC Plus jet nebulizer or Pari LC SPRINT jet nebulizer. In some embodiments, the nebulizer has an inlet pressure from about 10 to about 40 psig (e.g. 20-25 psig). In some embodiments, the inlet flow is from about 3 L/min to about 8 L/min (e.g. 5.2 L/min). In some embodiments, the exhaust air flow is from about 3 L/min to about 8 L/min (e.g. 5 L/min).

Actual dosage levels of the active ingredients in the pharmaceutical compositions can be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the subject being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount can include, but are not limited to, the severity of the subject's condition, the disorder being treated, the stability of the compound, and, if desired, another type of therapeutic agent being administered with the compound of the disclosure. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable dose of an active compound used in the compositions and methods of the disclosure will be that amount of the compound that is the lowest dose effective to produce a therapeutic effect. Such an effective dose will generally depend upon the factors described above.

### Numbered Embodiments

1. A method of treating an infection in a subject caused by nontuberculous Mycobacterium (NTM), the method comprising administering to the subject an effective amount of a bismuth-thiol (BT) composition that comprises a BT compound.
2. The method of embodiment 1, wherein the infection is a pulmonary infection.
3. The method of embodiment 1, wherein the infection is an extrapulmonary infection.
4. The method of any one of embodiments 1-3, wherein the NTM infection is caused by an antibiotic-resistant strain of NTM.
5. The method of any one of embodiments 1-4, wherein the NTM infection is caused by M. avium, M. avium subsp. hominissuis (MAH), M. abscessus, M. chelonae, M. bolletii, M. kansasii, M. ulcerans, M. avium complex (MAC) (M. avium and M. intracellulare), M. conspicuum, M. kansasii, M. peregrinum, M. immunogenum, M. xenopi, M. marinum, M. malmoense, M. marinum, M. mucogenicum, M. nonchromogenicum, M. scrofulaceum, M. simiae, M. smegmatis, M. szulgai, M. terrae, M. terrae complex, M. haemophilum, M. genavense, M. asiaticum, M. shimoidei, M. gordonae, M. nonchromogenicum, M. triplex, M. lentiflavum, M. celatum, M. fortuitum, M. fortuitum complex (M. fortuitum and M. chelonae), or a combination thereof.
6. The method of any one of embodiments 1-4, wherein the NTM infection is caused by M. abscessus, M. avium, or a combination thereof.
7. The method of any one of embodiments 1-4, wherein the NTM lung infection is caused by M. avium complex (M. avium and M. intracellulare).
8. The method of any one of embodiments 5-7, wherein the M. avium is M. avium subsp. hominissuis.
9. The method of any one of embodiments 1-8, wherein the NTM infection is a biofilm-associated NTM infection.
10. The method of any one of embodiments 1, 2 and 4-9, wherein the NTM infection is a chronic pulmonary infection.
11. The method of any one of embodiments 1-10, wherein the NTM infection is located in or on the lung mucosa, the bronchi, the alveoli, the macrophages, and/or the bronchioles.
12. The method of any one of embodiments 1-11, wherein the NTM infection is at least partially located in the macrophages.
13. The method of embodiment 11 or 12, wherein the macrophages are THP-1 macrophages.
14. The method of any one of embodiments 11-13, wherein upon administration of the BT composition to the subject, the bacterial load in the macrophages is reduced.
15. The method of any one of embodiments 11-14, wherein the macrophages are infected with one or more strains of M. abscessus and/or M. avium.
16. The method of any one of embodiments 1-15, wherein the NTM infection is resistant to treatment with amikacin.
17. The method of any one of embodiments 1-16, wherein the NTM infection is resistant to macrolide or azalide therapy.
18. The method of any one of embodiments 1-17, wherein the subject has a chronic lung condition.
19. The method of embodiment 18, wherein the chronic lung condition is the chronic lung condition is cystic fibrosis, chronic bronchitis, emphysema, bronchiectasis, pulmonary fibrosis, asbestosis, pneumonitis, chronic obstructive pulmonary disorder (COPD), or asthma.
20. The method of embodiment 18 or 19, wherein the chronic lung condition is cystic fibrosis.
21. The method of any one of embodiments 1-20, wherein the subject is administered about 30 μg to about 3,000 μg of BT compound per dose.
22. The method of embodiment 21, wherein the subject is administered about 100 μg to about 1,000 μg of BT compound per dose.
23. The method of any one of embodiments 1-22, wherein the BT composition is administered once per month, twice per month, three times per month, four times per month, once every two weeks, once per week, twice per week, or three times per week.
24. The method of any one of embodiments 1-23, wherein the BT composition is administered once or twice daily.
25. The method of any one of embodiments 1-24, wherein the BT compound is administered to the lungs of the subject.
26. The method of any one of embodiments 1-25, wherein the BT compound is administered by inhalation.
27. The method of any one of embodiments 1-26, wherein the BT composition is administered for a period of less than 24 months, less than 18 months, less than 12 months, less than 9 months, less than 6 months, less than 3 months, or less than 1 month.
28. The method of any one of embodiments 1-27, wherein the BT composition is administered for a period of 1 day to 56 days.
29. The method of any one of embodiments 1-27, wherein the BT composition is administered for a period of 14 days to 28 days.
30. The method of any one of embodiments 25-29, wherein the concentration of bismuth in the lungs after a single daily dose is from about 0.03 μg/g lung tissue to about 3 μg/g lung tissue.
31. The method of any one of embodiments 25-29, wherein the concentration of bismuth in the lungs after 28 daily doses is from about 0.3 μg/g lung tissue to about 60 μg/g lung tissue.
32. The method of any one of embodiments 1-31, wherein the BT compound is selected from BisBAL, BisEDT, Bis-dimercaprol, BisDTT, Bis-2-mercaptoethanol, BisDTE, BisPyr, BisEry, BisTol, BisBDT, BisPDT, BisPyr/BAL, BisPyr/BDT, BisPyr/EDT, BisPyr/PDT, BisPyr/Tol, BisPyr/Ery, bismuth-1-mercapto-2-propanol, BisEDT/CSTMN (1:1), BisPyr/CSTMN (1:1), BisBAL/CSTMN (1:1), BisTOL/CSTMN (1:1), and BisEDT/2-hydroxy-1-propanethiol.
33. The method of embodiment 32, wherein the BT compound is selected from BisEDT, BisBAL, BisPyr, BisEry, BisTol, BisBDT, or BisEDT/2-hydroxy-1-propane thiol.
34. The method of embodiment 32, wherein the BT compound is BisEDT or BisBAL.
35. The method of embodiment 32, wherein the BT compound is BisEDT.
36. The method of any one of embodiments 1-35, further comprising administering an effective amount of amikacin, clarithromycin, azithromycin, ethambutol, rifampicin, tigecycline, linezolid, imipenem, cefoxitin, or combination thereof to the subject in need.
37. A method of treating an NTM infection in a subject having a macrophage infection, comprising administering to the subject an effective amount of a bismuth-thiol (BT) composition that comprises a BT compound.
38. The method of embodiment 37, further comprising testing for the presence of bacteria-infected macrophages in a biological sample from the subject, and administering an effective amount of the bismuth-thiol (BT) composition to the subject if the sample tests positive for bacteria-infected macrophages.
39. The method of embodiment 37 or 38, wherein the macrophage cells are THP-1 cells.
40. The method of any one of embodiments 37-39, wherein the NTM infection is caused by M. avium, M. avium subsp. hominissuis (MAH), M. abscessus, M. chelonae, M. bolletii, M. kansasii, M. ulcerans, M. avium complex (MAC) (M. avium and M. intracellulare), M. conspicuum, M. kansasii, M. peregrinum, M. immunogenum, M. xenopi, M. marinum, M. malmoense, M. marinum, M. mucogenicum, M. nonchromogenicum, M. scrofulaceum, M. simiae, M. smegmatis, M. szulgai, M. terrae, M. terrae complex, M. haemophilum, M. genavense, M. asiaticum, M. shimoidei, M. gordonae, M. nonchromogenicum, M. triplex, M. lentiflavum, M. celatum, M. fortuitum, M. fortuitum complex (M. fortuitum and M. chelonae), or a combination thereof.
41. The method of any one of embodiments 37-40, wherein the NTM infection is caused by M. abscessus, M. avium, M. intracellulare, M. fortuitum, M. gordonae, M. kansasii, M. avium complex, M. marinum, M. terrae, M. cheloni, or a combination thereof.
42. The method of any one of embodiments 37-40, wherein the NTM infection is caused by M. abscessus, M. avium, or a combination thereof.
43. The method of any one of embodiments 37-40, wherein the NTM lung infection is caused by M. avium complex (M. avium and M. intracellulare).
44. The method of any one of embodiments 40-43, wherein the M. avium is M. avium subsp. hominissuis.
45. The method of any one of embodiments 37-44, wherein the NTM infection is a biofilm-associated NTM infection.
46. The method of any one of embodiments 37-45, wherein the BT compound is selected from BisBAL, BisEDT, Bis-dimercaprol, BisDTT, Bis-2-mercaptoethanol, BisDTE, BisPyr, BisEry, BisTol, BisBDT, BisPDT, BisPyr/BAL, BisPyr/BDT, BisPyr/EDT, BisPyr/PDT, BisPyr/Tol, BisPyr/Ery, bismuth-1-mercapto-2-propanol, BisEDT/CSTMN (1:1), BisPyr/CSTMN (1:1), BisBAL/CSTMN (1:1), BisTOL/CSTMN (1:1), and BisEDT/2-hydroxy-1-propanethiol.
47. The method of any one of embodiments 37-45, wherein the BT compound is BisEDT or BisBAL.
48. The method of any one of embodiments 37-45, wherein the BT compound is BisEDT.
49. The method of any one of embodiments 37-48, wherein the BT composition is administered once per month, twice per month, three times per month, four times per month, once every two weeks, once per week, twice per week, or three times per week.
50. The method of any one of embodiments 37-48, wherein the BT composition is administered once or twice daily.
51. The method of any one of embodiments 37-50, wherein the BT composition is administered for a period of less than 24 months, less than 18 months, less than 12 months, less than 9 months, less than 6 months, less than 3 months, or less than 1 month.
52. The method of any one of embodiments 37-51, wherein the BT compound is administered for a period of 1 day to 56 days.
53. The method of any one of embodiments 37-52, wherein the BT compound is administered for a period of 14 days to 28 days.
54. The method of any one of embodiments 37-52, further comprising administering an effective amount of amikacin, clarithromycin, azithromycin, ethambutol, rifampicin, tigecycline, linezolid, imipenem, cefoxitin, or combination thereof to the subject in need thereof.
55. The method of any one of embodiments 37-53, further comprising administering amikacin to the subject in need thereof.
56. The method of embodiment 55, wherein an effective amount of amikacin, clarithromycin, azithromycin, ethambutol, rifampicin, tigecycline, linezolid, imipenem, cefoxitin, or combination thereof is an amount that is ineffective in treating the NTM infection when administered without a BT composition.
57. The method of embodiment 55, wherein administration of an effective amount of the BT composition and an effective amount of amikacin results in a synergistic effect in treating the NTM infection.
58. The method of any one of embodiments 37-57, wherein the subject has a chronic lung condition.
59. The method of embodiment 58, wherein the chronic lung condition is the chronic lung condition is cystic fibrosis, chronic bronchitis, emphysema, bronchiectasis, pulmonary fibrosis, asbestosis, pneumonitis, chronic obstructive pulmonary disorder (COPD), or asthma.
60. The method of any one of embodiment 58 or 59, wherein the chronic lung condition is cystic fibrosis.
61. The method of any one of embodiments 37-60, wherein the subject is administered about 30 μg to about 3,000 μg of BT compound per day.
62. The method of any one of embodiments 37-60, wherein the subject is administered about 100 μg to about 1,000 μg of BT compound per day.
63. The method of any one of embodiments 37-62, wherein the BT composition is administered to the lungs of the subject.
64. The method of any one of embodiments 37-63, wherein the BT composition is administered by inhalation.
65. The method of embodiment 63 or 64, wherein the concentration of bismuth in the lungs after a single daily dose is from about 0.03 μg/g lung tissue to about 3 μg/g lung tissue.
66. The method of any one of embodiments 63-65, wherein the concentration of bismuth in the lungs after 28 daily doses is from about 0.3 μg/g lung tissue to about 60 μg/g lung tissue.
67. The method of any one of embodiments 37-66, wherein the subject in need of treatment was previously unresponsive to NTM therapy.
68. The method of any one of embodiments 37-67, wherein the subject in need of treatment was previously unresponsive to amikacin.
69. A method of reducing NTM intracellular bacterial burden in a subject, comprising contacting an infected cell of the subject with an effective amount of a bismuth-thiol (BT) composition that comprises a BT compound.
70. The method of embodiment 69, wherein the cell is infected by M. avium, M. avium subsp. hominissuis (MAH), M. abscessus, M. chelonae, M. bolletii, M. kansasii, M. ulcerans, M. avium complex (MAC) (M. avium and M. intracellulare), M. conspicuum, M. kansasii, M. peregrinum, M. immunogenum, M. xenopi, M. marinum, M. malmoense, M. marinum, M. mucogenicum, M. nonchromogenicum, M. scrofulaceum, M. simiae, M. smegmatis, M. szulgai, M. terrae, M. terrae complex, M. haemophilum, M. genavense, M. asiaticum, M. shimoidei, M. gordonae, M. nonchromogenicum, M. triplex, M. lentiflavum, M. celatum, M. fortuitum, M. fortuitum complex (M. fortuitum and M. chelonae), or a combination thereof.
71. The method of embodiment 69 or 70, wherein the cell is infected by M. abscessus, M. avium, M. intracellulare, M. fortuitum, M. gordonae, M. kansasii, M. avium complex, M. marinum, M. terrae, M. cheloni, or a combination thereof.
72. The method of any one of embodiments 69-71, wherein the cell is infected by M. abscessus, M. avium, or a combination thereof.
73. The method of any one of embodiments 69-71, wherein the cell is infected by M. avium complex (M. avium and M. intracellulare).
74. The method of any one of embodiments 70-73, wherein the M. avium infection is a M. avium subsp. hominissuis infection.
75. The method of any one of embodiments 69-74, wherein the infected cell is a macrophage.
76. The method of embodiment 75, wherein the macrophage is a THP-1 μmacrophage.
77. The method of any one of embodiments 69-76, wherein contacting the cell with the BT composition results in phagocytosis of the BT compound.
78. The method of embodiment 77, wherein the intracellular bacterial burden is reduced by about 10-fold to about 1000-fold.
79. The method of embodiment 77 or 78, wherein the intracellular bacterial burden is reduced by about 10-fold, about 50-fold, about 100-fold, about 250-fold, about 500-fold, or about 1000-fold.
80. The method of any one of embodiments 69-76, wherein contacting the cell with the BT composition keeps the intracellular bacterial burden from increasing.
81. The method of any one of embodiments 75-80, wherein the macrophages are infected with one or more strains of M. abscessus.
82. The method of any one of embodiments 75-80, wherein the macrophages are infected with one or more strains of M. avium.
83. The method of any one of embodiments 69-82, wherein the BT composition comprises a BT compound selected from the group consisting of BisBAL, BisEDT, Bis-dimercaprol, BisDTT, Bis-2-mercaptoethanol, Bis-DTE, BisPyr, BisEry, BisTol, BisBDT, BisPDT, BisPyr/BAL, BisPyr/BDT, BisPyr/EDT, BisPyr/PDT, BisPyr/Tol, BisPyr/Ery, bismuth-1-mercapto-2-propanol, BisEDT/CSTMN (1:1), BisPyr/CSTMN (1:1), BisBAL/CSTMN (1:1), BisTOL/CSTMN (1:1), and BisEDT/2-hydroxy-1-propanethiol.
84. The method of any one of embodiments 69-82, wherein the BT compound is selected from the group consisting of BisEDT, BisBAL, BisPyr, BisEry, BisTol, BisBDT, or BisEDT/2-hydroxy-1-propane thiol.
85. The method of any one of embodiments 69-82, wherein the BT compound is BisEDT or BisBAL.
86. The method of any one of embodiments 69-82, wherein the BT compound is BisEDT.
87. The method of any one of embodiments 69-86, where the BT compound exhibits a bacteriostatic effect.
88. The method of any one of embodiments 69-86, wherein the BT compound exhibits a bactericidal effect.
89. The method of any one of embodiments 69-88, wherein the subject has a lung infection.
90. The method of embodiment 89, wherein the lung infection is a chronic lung condition.
91. The method of embodiment 90, wherein the chronic lung condition is the chronic lung condition is cystic fibrosis, chronic bronchitis, emphysema, bronchiectasis, pulmonary fibrosis, asbestosis, pneumonitis, chronic obstructive pulmonary disorder (COPD), or asthma.
92. The method of embodiment 90, wherein the chronic lung condition is cystic fibrosis.
93. The method of any one of embodiments 69-92, wherein the subject is administered about 30 μg to about 3,000 μg of BT compound per day.
94. The method of any one of embodiments 69-92, wherein the subject is administered about 100 μg to about 1,000 μg of BT compound per day.
95. The method of any one of embodiments 69-94, wherein the BT composition is administered once per month, twice per month, three times per month, four times per month, once every two weeks, once per week, twice per week, or three times per week.
96. The method of any one of embodiments 69-95, wherein the BT composition is administered once or twice daily.
97. The method of any one of embodiments 69-96, wherein the BT composition is administered to the lungs of the subject.
98. The method of any one of embodiments 69-97, wherein the BT composition is administered by inhalation.
99. The method of any one of embodiments 69-98, wherein the BT composition is administered for a period of less than 24 months, less than 18 months, less than 12 months, less than 9 months, less than 6 months, less than 3 months, or less than 1 month.
100. The method of any one of embodiments 69-98, wherein the BT composition is administered for a period of 1 day to 56 days.
101. The method of any one of embodiments 69-98, wherein the BT composition is administered for a period of 14 days to 28 days.
102. The method of any one of embodiments 97-101, wherein the concentration bismuth in the lungs after a single daily dose is from about 0.03 μg/g lung tissue to about 3 μg/g lung tissue.
103. The method of any one of embodiments 97-101, wherein the concentration of bismuth in the lungs after 28 daily doses is from about 0.3 μg/g lung tissue to about 60 μg/g lung tissue.
104. The method of any one of embodiments 69-103, further comprising contacting the infected cell of the subject with amikacin.
105. The method of embodiment 104, wherein contracting the infected cell with a combination of a BT composition and amikacin exhibits a synergistic effect in reducing intracellular bacterial burden.
106. A method for treating or providing prophylaxis against a nontuberculous Mycobacterium (NTM) lung infection in a subject in need of treatment or prophylaxis, comprising:
   administering to the lungs of the subject for an administration period, a BT composition comprising a BT compound.
107. The method of embodiment 106, wherein administering to the lungs of the patient comprises aerosolizing the BT composition to provide an aerosolized BT composition, and administering the aerosolized BT composition to the lungs of the subject.
108. The method of embodiment 107, wherein administering the aerosolized BT composition to the lungs of the subject is by a nebulizer, dry powder inhalation, nanoparticle inhalation, or metered-dose inhalation.
109. The method of any one of embodiments 106-108, wherein the aerosolized BT composition is administered once per day in a single dosing session during the administration period.
110. The method of embodiment 109, wherein during the single dosing session, the aerosolized BT composition is administered in less than about 75 minutes, less than about 60 minutes, less than about 30 minutes, less than about 15 minutes, or less than about 5 minutes.
111. The method of embodiment 106, wherein during the single dosing session, the aerosolized BT composition is administered in about 60 to about 75 minutes, about 45 minutes to about 60 minutes, about 30 minutes to about 45 minutes, about 20 minutes to about 30 minutes, or about 15 minutes to about 20 minutes.
112. The method of any one of embodiments 106-111, wherein the aerosolized BT composition is administered over a 1 week, 2 week, 3 week, 4 week, 1 month, 2 month, 3 month, 4 month, 5 month, or 6 month treatment period.
113. The method of any one of embodiments 106-111, wherein the aerosolized BT composition is administered over a 6 month treatment period.
114. The method of any one of embodiments 106-111, wherein the subject in need of treatment of prophylaxis has cystic fibrosis, bronchiectasis, chronic obstructive pulmonary disorder (COPD), or asthma.
115. The method of any one of embodiments 106-111, wherein the subject in need of treatment or prophylaxis has cystic fibrosis.
116. The method of any one of embodiments 106-115, wherein the subject in need of treatment or prophylaxis was previously unresponsive to NTM therapy.
117. The method of any one of embodiments 106-116, wherein the subject in need of treatment or prophylaxis was previously unresponsive to amikacin.
118. The method of any one of embodiments 106-117, wherein the subject in need of treatment or prophylaxis has a co-morbid condition selected from the group consisting of diabetes, mitral valve disorder, acute bronchitis, pulmonary hypertension, pneumonia, asthma, trachea cancer, bronchus cancer, lung cancer, cystic fibrosis, pulmonary fibrosis, a larynx anomaly, a trachea anomaly, a bronchus anomaly, aspergillosis, HIV and bronchiectasis.
119. The method of any one of embodiments 106-117, wherein the NTM lung infection is M. avium, M. avium subsp. hominissuis (MAH), M. abscessus, M. chelonae, M. bolletii, M. kansasii, M. ulcerans, M. avium, M. avium complex (MAC) (M. avium and M. intracellulare), M. conspicuum, M. kansasii, M. peregrinum, M. immunogenum, M. xenopi, M. marinum, M. malmoense, M. marinum, M. mucogenicum, M. nonchromogenicum, M. scrofulaceum, M. simiae, M. smegmatis, M. szulgai, M. terrae, M. terrae complex, M. haemophilum, M. genavense, M. asiaticum, M. shimoidei, M. gordonae, M. nonchromogenicum, M. triplex, M. lentiflavum, M. celatum, M. fortuitum, M. fortuitum complex (M. fortuitum and M. chelonae), or a combination thereof.
120. The method of any one of embodiments 106-117, wherein the NTM lung infection is a M. avium infection.
121. The method of embodiment 120, wherein the M. avium infection is a M. avium subsp. hominissuis infection.
122. The method of any one of embodiments 106-117, wherein the NTM lung infection is M. avium complex (M. avium and M. intracellulare).
123. The method of any one of embodiments 106-117, wherein the NTM lung infection is a M. abscessus infection.
124. The method of any one of embodiments 106-123, wherein the NTM lung infection is a macrolide resistant NTM lung infection.
125. The method of any one of embodiments 106-124, wherein the NTM lung infection is a biofilm-associated infection.
126. The method of any one of embodiments 106-124, wherein during the administration period, or subsequent to the administration period, the subject exhibits an NTM culture conversion to negative.
127. The method of embodiment 126, wherein the time to NTM culture conversion to negative is about 10 days, about 20 days, about 30 days, about 40 days, about 50 days, about 60 days, about 70 days, about 80 days, about 90 days, about 100 days or about 110 days.
128. The method of embodiment 126, wherein the time to NTM culture conversion to negative is from about 20 days to about 200 days, from about 20 days to about 190 days, from about 20 days to about 180 days, from about 20 days to about 160 days, from about 20 days to about 150 days, from about 20 days to about 140 days, from about 20 days to about 130 days, from about 20 days to about 120 days, from about 20 days to about 110 days, from about 30 days to about 110 days, or from about 30 days to about 100 days.
129. The method of any one of embodiments 106-128, wherein the subject experiences an improvement in FEV₁ for at least 15 days after the administration period ends, as compared to the FEV₁ of the subject prior to the administration period.
130. The method of any one of embodiments 106-129, wherein the subject experiences an improvement in blood oxygen saturation for at least 15 days after the administration period ends, as compared to the blood oxygen saturation of the subject prior to the administration period.
131. The method of embodiment 129, wherein the subject's FEV₁ is increased at least 5% over the FEV₁ of the subject prior to the administration period.
132. The method of embodiment 129, wherein the subject's FEV₁ is increased at least 10% over the FEV₁ of the subject prior to the administration period.
133. The method of embodiment 129, wherein the subject's FEV₁ is increased at least 15% over the FEV₁ of the subject prior to the administration period.
134. The method of embodiment 129, wherein the subject's FEV₁ is increased by 5% to 50% over the FEV₁ prior to the administration period.
135. The method of any one of embodiments 106-134, wherein the subject exhibits an increased number of meters walked in the 6 μminute walk test (6MWT), as compared to the number of meters walked by the subject prior to undergoing the treatment method.
136. The method of embodiment 135, wherein the increased number of meters walked in the 6MWT is at least about 5 meters.
137. The method of embodiment 135, wherein the increased number of meters walked in the 6MWT is at least about 10 meters.
138. The method of embodiment 135, wherein the increased number of meters walked in the 6MWT is from about 5 meters to about 50 meters.
139. The method of embodiment 135, wherein the increased number of meters walked in the 6MWT is from about 15 meters to about 50 meters.
140. The method of any one of embodiment 106-139, wherein the BT composition comprises a BT compound selected from the group consisting of BisBAL, BisEDT, Bis-dimercaprol, BisDTT, Bis-2-mercaptoethanol, Bis-DTE, BisPyr, BisEry, BisTol, BisBDT, BisPDT, BisPyr/BAL, BisPyr/BDT, BisPyr/EDT, BisPyr/PDT, Bis-Pyr/Tol, BisPyr/Ery, bismuth-1-mercapto-2-propanol, BisEDT/CSTMN (1:1), BisPyr/CSTMN (1:1), BisBAL/CSTMN (1:1), BisTOL/CSTMN (1:1), and BisEDT/2-hydroxy-1-propanethiol.
141. The method of any one of embodiments 106-139, wherein the BT compound is selected from the group consisting of BisEDT, BisBAL, BisPyr, BisEry, BisTol, BisBDT, or BisEDT/2-hydroxy-1-propane thiol.
142. The method of any one of embodiments 106-139, wherein the BT compound is BisEDT or BisBAL.
143. The method of any one of embodiments 106-139, wherein the BT compound is BisEDT.
144. The method of any one of embodiments 106-143, wherein the concentration of bismuth in the lungs after a single daily dose is from about 0.03 μg/g lung tissue to about 3 μg/g lung tissue.
145. A method for treating a biofilm-associated nontuberculous Mycobacterium (NTM) infection in the lungs of a subject in need, comprising administering to the subject a BT composition comprising a BT compound.
146. The method of embodiment 145, wherein the subject has a chronic lung condition.
147. The method of embodiment 146, wherein the chronic lung condition is the chronic lung condition is cystic fibrosis, chronic bronchitis, emphysema, bronchiectasis, pulmonary fibrosis, asbestosis, pneumonitis, chronic obstructive pulmonary disorder (COPD), or asthma.
148. The method of embodiment 145 or 146, wherein the chronic lung condition is cystic fibrosis.
149. The method of any one of embodiments 145-148, wherein the biofilm is caused by an antibiotic-resistant strain of a microorganism.
150. The method of any one of embodiments 145-149, wherein the NTM lung infection is M. avium, M. avium subsp. hominissuis (MAH), M. abscessus, M. chelonae, M. bolletii, M. kansasii, M. ulcerans, M. avium, M. avium complex (MAC) (M. avium and M. intracellulare), M. conspicuum, M. kansasii, M. peregrinum, M. immunogenum, M. xenopi, M. marinum, M. malmoense, M. marinum, M. mucogenicum, M. nonchromogenicum, M. scrofulaceum, M. simiae, M. smegmatis, M. szulgai, M. terrae, M. terrae complex, M. haemophilum, M. genavense, M. asiaticum, M. shimoidei, M. gordonae, M. nonchromogenicum, M. triplex, M. lentiflavum, M. celatum, M. fortuitum, M. fortuitum complex (M. fortuitum and M. chelonae), or a combination thereof.
151. The method of any one of embodiments 145-149, wherein the NTM lung infection is a M. avium infection.
152. The method of any one of embodiments 145-149, wherein the NTM lung infection is M. avium complex (M. avium and M. intracellulare).
153. The method of any one of embodiments 150-152, wherein the M. avium infection is a M. avium subsp. hominissuis infection.
154. The method of any one of embodiments 145-149, wherein the NTM lung infection is a M abscessus infection.
155. The method of any one of embodiments 145-154, wherein the BT composition comprises a BT compound selected from the group consisting of BisBAL, BisEDT, Bis-dimercaprol, BisDTT, Bis-2-mercaptoethanol, Bis-DTE, BisPyr, BisEry, BisTol, BisBDT, BisPDT, BisPyr/BAL, BisPyr/BDT, BisPyr/EDT, BisPyr/PDT, Bis-Pyr/Tol, BisPyr/Ery, bismuth-1-mercapto-2-propanol, BisEDT/CSTMN (1:1), BisPyr/CSTMN (1:1), BisBAL/CSTMN (1:1), BisTOL/CSTMN (1:1), and BisEDT/2-hydroxy-1-propanethiol.
156. The method of any one of embodiments 145-154, wherein the BT compound is selected from the group consisting of BisEDT, BisBAL, BisPyr, BisEry, BisTol, BisBDT, or BisEDT/2-hydroxy-1-propane thiol.
157. The method of any one of embodiments 145-154, wherein the BT compound is BisEDT or BisBAL.
158. The method of any one of embodiments 145-154, wherein the BT compound is BisEDT.
159. The method of any one of embodiments 145-158, wherein the subject is administered about 30 μg to about 3,000 μg of BT compound per day.
160. The method of any one of embodiments 145-158, wherein the subject is administered about 100 μg to about 1,000 μg of BT compound per day.
161. The method of any one of embodiments 145-160, wherein the BT composition is administered once per month, twice per month, three times per month, four times per month, once every two weeks, once per week, twice per week, or three times per week.
162. The method of any one of embodiments 145-160, wherein the BT composition is administered once or twice daily.
163. The method of any one of embodiments 145-162, wherein the BT compound is administered to the lungs of the subject.
164. The method of any one of embodiments 145-163, wherein the BT compound is administered by inhalation.
165. The method of any one of embodiments 145-164, wherein the BT composition is administered for a period of less than 24 months, less than 18 months, less than 12 months, less than 9 months, less than 6 months, less than 3 months, or less than 1 month.
165. The method of any one of embodiments 145-164, wherein the BT composition is administered for a period of 1 day to 56 days.
166. The method of any one of embodiments 145-164, wherein the BT composition is administered for a period of 14 days to 28 days.
167. The method of any one of embodiments 145-166, wherein the concentration of bismuth in the lungs after a single daily dose is from about 0.03 μg/g lung tissue to about 3 μg/g lung tissue.
168. The method of any one of embodiments 145-166, wherein the concentration of bismuth in the lungs after 28 daily doses is from about 0.3 μg/g lung tissue to about 60 μg/g lung tissue.
169. A method of treating an NTM infection in a subject, the method comprising:
   (i) testing for the presence of bacteria-infected macrophages in a biological sample from the subject; and
   (ii) administering an effective amount of the bismuth-thiol (BT) composition that comprises a BT compound to the subject if the sample tests positive for bacteria-infected macrophages.
170. The method of embodiment 169, wherein the NTM infection is a lung infection.
171. The method of embodiment 169 or 170, wherein the macrophages are tested for the presence of M. avium, M. avium subsp. hominissuis (MAH), M. abscessus, M. chelonae, M. bolletii, M. kansasii, M. ulcerans, M. avium complex (MAC) (M. avium and M. intracellulare), M. chimaera, M. conspicuum, M. peregrinum, M. immunogenum, M. xenopi, M. marinum, M. malmoense, M. mucogenicum, M. nonchromogenicum, M. scrofulaceum, M. simiae, M. smegmatis, M. szulgai, M. terrae, M. terrae complex, M. haemophilum, M. genavense, M. gordonae, M. fortuitum, M. fortuitum complex (M. fortuitum and M. chelonae), or a combination thereof.
172. The method of any one of embodiments 169-171, wherein the macrophages are tested for the presence of M. abscessus, M. avium, M. intracellulare, M. fortuitum, M. gordonae, M. kansasii, M. avium complex, M. marinum, M. terrae, M. cheloni, or a combination thereof.
173. The method of any one of embodiments 169-171, wherein the macrophages are tested for the presence of M. abscessus, M. avium, or a combination thereof.
174. The method of any one of embodiments 169-171, wherein the macrophages are tested for the presence of M. avium complex (M. avium and M. intracellulare).
175. The method of any one of embodiments 171-174, wherein the M. avium infection is a M. avium subsp. hominissuis infection.
176. The method of any one of embodiments 169-171, wherein the macrophages are tested for the presence of M. abscessus.
177. The method of any one of embodiments 169-176, wherein the subject is administered about 30 μg to about 3,000 μg of BT compound per day.
178. The method of any one of embodiments 169-176, wherein the subject is administered about 100 μg to about 1,000 μg of BT compound per day.
179. The method of any one of embodiments 169-178, wherein the BT composition is administered once per month, twice per month, three times per month, four times per month, once every two weeks, once per week, twice per week, or three times per week.
180. The method of any one of embodiments 169-179, wherein the BT composition is administered once or twice daily.
181. The method of any one of embodiments 169-180, wherein the BT composition is administered to the lungs of the subject.
182. The method of any one of embodiments 169-181, wherein the BT compound is administered by inhalation.
183. The method of any one of embodiments 169-182, wherein the BT composition is administered for a period of less than 24 months, less than 18 months, less than 12 months, less than 9 months, less than 6 months, less than 3 months, or less than 1 month.
184. The method of any one of embodiments 169-182, wherein the BT composition is administered for a period of 1 day to 56 days.
185. The method of any one of embodiments 169-182, wherein the BT composition is administered for a period of 14 days to 28 days.
186. The method of any one of embodiments 169-185, wherein the concentration of bismuth in the lungs after a single daily dose is from about 0.03 μg/g lung tissue to about 3 μg/g lung tissue.
187. The method of any one of embodiments 169-186, wherein the concentration of bismuth in the lungs after 28 daily doses is from about 0.3 μg/g lung tissue to about 60 μg/g lung tissue.
188. The method of any one of embodiments 169-187, wherein the BT compound is selected from the group consisting of BisBAL, BisEDT, Bis-dimercaprol, BisDTT, Bis-2-mercaptoethanol, Bis-DTE, BisPyr, BisEry, BisTol, BisBDT, BisPDT, BisPyr/BAL, BisPyr/BDT, BisPyr/EDT, BisPyr/PDT, Bis-Pyr/Tol, BisPyr/Ery, bismuth-1-mercapto-2-propanol, BisEDT/CSTMN (1:1), BisPyr/CSTMN (1:1), BisBAL/CSTMN (1:1), BisTOL/CSTMN (1:1), and BisEDT/2-hydroxy-1-propanethiol.
189. The method of any one of embodiments 169-187, wherein the BT compound is selected from the group consisting of BisEDT, BisBAL, BisPyr, BisEry, BisTol, BisBDT, or BisEDT/2-hydroxy-1-propane thiol.
190. The method of any one of embodiments 169-187, wherein the BT compound is BisEDT or BisBAL.
191. The method of any one of embodiments 169-187, wherein the BT compound is BisEDT.
192. The method of any one of embodiments 1-191, wherein, the pharmaceutical composition comprises a BT compound suspended therein, and one or more pharmaceutically acceptable excipients, wherein the composition is formulated to administer the BT compound topically to the lungs of a subject.
193. The method of embodiment 192, wherein the composition comprises a plurality of particles comprising the BT compound.
194. The method of embodiment 193, wherein the BT composition comprises the BT compound at a concentration greater than about 0.1 mg/mL, about 0.05% to about 1.0% of polysorbate 80, about 0.05 mM to 40 mM of sodium chloride, and optionally about 2 mM to 20 mM of sodium phosphate at about pH 7.4. 195. The method of embodiment 194, wherein the BT compound is BisEDT.
196. The method of any one of embodiments 192-195, wherein topical administration to the lungs of the subject comprises aerosolizing the BT composition to provide an aerosolized BT composition, and administering the aerosolized BT composition to the lungs of the subject.
197. The method of embodiment 196, wherein administering the aerosolized BT composition to the lungs of the subject is by a nebulizer.
198. The method of embodiment 196 or 197, wherein the aerosolized BT composition is administered once per day in a single dosing session during the administration period.
199. The method of embodiment 198, wherein during the single dosing session, the aerosolized BT composition is administered in less than about 75 minutes, less than about 60 minutes, less than about 30 minutes, less than about 15 minutes, or less than about 5 minutes.
200. The method of embodiment 198, wherein during the single dosing session, the aerosolized BT composition is administered in about 60 to about 75 minutes, about 45 minutes to about 60 minutes, about 30 minutes to about 45 minutes, about 20 minutes to about 30 minutes, or about 15 minutes to about 20 minutes.

### EXAMPLES

The following examples are provided to illustrate the present disclosure, and should not be construed as limiting thereof.

### Example 1: Representative Synthesis of BT Compounds

Representative synthesis of BisEDT at 20° C. with 1.25-hour addition of thiol via syringe pump, and polypropylene cloth for filtration: BisEDT synthesis was performed on 10-g scale. To a 1-L jacketed reactor was charged USP water (480 mL, 48 vol), followed by 70% HNO3 (34 mL, 3.4 vol). A solution of bismuth subnitrate (10 g, 6.84 mmol) in water (43 mL, 4.3 vol) and 70% HNO3 (14 mL, 1.4 vol) was added at 20° C. The reaction mixture was cooled to 15° C. for addition of 95% Ethanol. The 95% ethanol (180 mL, 18 vol) was then added slowly. (Ethanol addition is exothermic, temperature reached 22° C.). The temperature was then adjusted back to 20° C. This was followed by dropwise addition of 1,2 ethanedithiol (4.3 mL, 7.5 mmol in 95% ethanol in 94 mL, 9.4 vol) over a period of 1.25 hour with the batch temperature at 20° C. during which time it turned into a yellow suspension. The reaction was stirred at 20° C. overnight. The reaction mixture was filtered through polypropylene cloth and washed with 95% ethanol (45 mL, 4.5 vol). The wet cake was charged back to the reactor and slurried in 95% ethanol (380 mL, 38 vol) for two hours at 20° C. The suspension was then filtered (same cloth) and washed with 95% ethanol (30 mL, 3 vol). The wet cake was again slurried in 95% EtOH (170 mL, 17 vol) at 20° C., filtered (same cloth), and washed with 95% ethanol (30 mL, 3 vol). The wet cake was then slurried in acetone (170 mL, 17 vol) at 20° C. overnight, followed by filtration (same cloth) and acetone wash (20 mL, 2 vol). The acetone (170 ml, 17 vol) treatment was repeated on the solids and stirred for 2 hours. The suspension was filtered (same cloth) and washed with acetone (30 mL, 3 vol) and died at 45° C. and dried at 45° C. (18 hours) to provide canary yellow solid (10.81 g 91.0%).

Representative synthesis of BisEDT at 15° C. with 1 hour addition of thiol via syringe pump, and polypropylene cloth for filtration: The synthesis BisEDT was performed on 10-g scale, temperature profile was studied with data logger. Ethane dithiol was added at 15° C. over 1 hour via syringe pump and the filtration was performed using PP filter cloth. To a 1-L jacketed reactor was charged USP water (480 mL, 48 vol) and cooled to 15° C., followed by 70% HNO3 (34 mL, 3.4 vol). A solution of bismuth subnitrate (10 g, 6.84 mmol) in water (43 mL, 4.3 vol) and 70% HNO3 (14 mL, 1.4 vol) was added at the same temperature. The 95% ethanol (180 mL, 18 vol) was then added slowly. (Ethanol addition is exothermic, temperature reached 22.5° C.). It was then allowed to cool to 15° C. This was followed by dropwise addition of 1,2 ethanedithiol (4.3 mL, 7.5 mmol in 95% ethanol in 94 mL, 9.4 vol) over an hour with the batch temperature at 15° C. The reaction was allowed to stir at 15° C. overnight. The reaction mixture was filtered through polypropylene cloth and washed with 95% ethanol (45 mL, 4.5 vol). The wet cake was charged back to the reactor and slurried in 95% ethanol (380 mL, 38 vol) for two hours at 20° C. The suspension was then filtered (same cloth) and washed with 95% ethanol (30 mL, 3 vol). The wet cake was again slurried in 95% EtOH (170 mL, 17 vol) at 20° C., filtered (same cloth), and washed with 95% ethanol (30 mL, 3 vol). The wet cake was then slurried in acetone (170 mL, 17 vol) at 20° C. overnight, followed by filtration (same cloth) and acetone wash (20 mL, 2 vol). The acetone (170 ml, 17 vol) treatment was repeated on the solids and stirred for 2 hours. The suspension was filtered (same cloth) and washed with acetone (30 mL, 3 vol) and died at 45° C. and dried at 45° C. (18 hours) to provide canary yellow solid (10.43 g 87.8%).

### Example 2: MIC Activity of BisEDT and Comparators Against M. avium and M. abscessus Bacterial Strains

### Study Design

Mycobacteria strains were used to determine MICs of BisEDT and amikacin. Strains included cystic fibrosis patient isolates from National Jewish Hospital. A range of high dose to low dose of antibiotics was used to determine bacteria susceptibility. M. avium strain 104, M. avium strain 3388, and CF patient strain DNA00133 were grown to log phase (7 days) while M. abscessus strain 19977, CF patient strain DNA00703, and CF patient strain DNA01715 achieved log phase (4 days) on 7H10 middlebrook media plates supplemented with 10% OADC (Oleic acid, Albumin, Dextrose, Catalase). For amikacin, 128 μg/ml was added to 1 ml of 7H9 middlebrook broth supplemented with 10% OADC then diluted 1:1 until the concentration reached 1 μg/ml. For BisEDT, two types of compound physical states were tested. First, BisEDT was solubilized in DMSO (furthermore labeled soluble BisEDT). Second, BisEDT was made in suspension to mimic in vivo studies (furthermore labeled insoluble BisEDT). For each type of BisEDT, 16 μg/ml was added to 1 ml of 7H9 broth supplemented with OADC then diluted 1:1 until the concentrations reached 0.125 μg/ml. Control tubes containing no antibiotics were also included for each antibiotic tested. Inoculums of 109 bacteria for each strain was measured with optical density (O.D.) at 595 nm. 10 μl of these inoculums were added to each antibiotic tube and incubated in a shaking incubator at 37° C. and 200 rpm. Slow growing stains were measured for growth after 10 days incubation while fast growing strains were incubated for 5 days. After incubation, 100 μl from each tube was pulled and placed in a 96-well plate for O.D. reading. Turbidity determined sensitive, intermediate, or resistant bacteria.

### MIC Activity in Mycobacterial Strains:

For M. avium strains, the amikacin MICs were determined to be 8 μg/ml, except the CF patient strain (DNA00133) which was resistant to 128 μg/ml. Soluble BisEDT inhibited all strains at 4 μg/ml, while insoluble BisEDT showed inhibition for the amikacin resistant patient strain DNA0133 at 8 μg/ml (Table 1).

TABLE 1
Sensitivity of BisEDT and comparator against M. avium strains.

| amikacin | | MIC μg/mL | | | | | | | |
|---|---|---|---|---|---|---|---|---|---|
| Strain | 128 | 64 | 32 | 16 | 8 | 4 | 2 | 1 | 0 |
| MAH 104 | S | S | S | S | S | R | R | R | R |
| MAH 3388 | S | S | S | S | S | I | R | R | R |
| DNA00133 | R | R | R | R | R | R | R | R | R |

| Insoluble BisEDT | | MIC μg/mL | | | | | | | |
|---|---|---|---|---|---|---|---|---|---|
| | 16 | 8 | 4 | 2 | 1 | 0.5 | 0.25 | 0.125 | 0 |
| MAH 104 | R | R | R | R | R | R | R | R | R |
| MAH 3388 | R | R | R | R | R | R | R | R | R |
| DNA00133 | S | S | I | R | R | R | R | R | R |

| BisEDT in DMSO | | MIC μg/mL | | | | | | | |
|---|---|---|---|---|---|---|---|---|---|
| Strain | 16 | 8 | 4 | 2 | 1 | 0.5 | 0.25 | 0.125 | 0 |
| MAH 104 | S | S | S | I | R | R | R | R | R |
| MAH 3388 | S | S | S | R | R | R | R | R | R |
| DNA00133 | S | S | S | S | I | R | R | R | R |

| Strain | Strain Characteristics | Phenotype |
|---|---|---|
| MAH 104 | Wildtype (bacteremia) | R = resistant |
| MAH 3388 | Lung Infection Isolate | I = intermediate |
| DNA00133 | CF Isolate | S = sensitive |

For M. abscessus strains, there was variable inhibition by amikacin. Strain 19977 was sensitive at 32 μg/ml, strain from patient 00703 was sensitive at 64 μg/ml, and strain from patient 01715 was sensitive at 16 μg/ml. As shown by the data in Table 2, all M. abscessus strains were more sensitive to BisEDT formulations (1-4 μg/ml) than amikacin.

TABLE 2
Sensitivity of BisEDT and comparator against M. abscessus strains.

| amikacin | | MIC μg/mL | | | | | | | |
|---|---|---|---|---|---|---|---|---|---|
| Strain | 128 | 64 | 32 | 16 | 8 | 4 | 2 | 1 | 0 |
| 19977 | S | S | S | R | R | R | R | R | R |
| DNA00703 | S | S | I | I | R | R | R | R | R |
| DNA01715 | S | S | S | S | I | R | R | R | R |

| Insoluble BisEDT | | MIC μg/mL | | | | | | | |
|---|---|---|---|---|---|---|---|---|---|
| | 16 | 8 | 4 | 2 | 1 | 0.5 | 0.25 | 0.125 | 0 |
| 19977 | S | S | S | S | S | R | R | R | R |
| DNA00703 | S | S | S | I | R | R | R | R | R |
| DNA01715 | S | S | S | S | S | I | R | R | R |

| BisEDT in DMSO | | MIC μg/mL | | | | | | | |
|---|---|---|---|---|---|---|---|---|---|
| 19977 | 16 | 8 | 4 | 2 | 1 | 0.5 | 0.25 | 0.125 | 0 |
| DNA00703 | S | S | S | S | S | I | R | R | R |
| DNA01715 | S | S | S | S | I | R | R | R | R |
| 19977 | S | S | S | S | S | I | R | R | R |

| | Strain | |
|---|---|---|
| Strain | Characteristics | Phenotype |
| 19977 | Wildtype | R = resistant |
| DNA00703 | Patient strain | I = intermediate |
| DNA01715 | Patient strain | S = sensitive |

Conclusions: BisEDT demonstrated improved in vitro activity, including MICs for the treatment of M. abscessus infections that were 16- to 32-times lower compared to amikacin.

### Example 3: BisEDT Treatment of THP-1 Macrophages Infected with M. avium or M. abscessus

### Objective

To investigate the efficacy of BisEDT in reducing M. avium and M. abscessus levels in vitro.

### Study Design

THP-1 macrophages were differentiated with 50 ng/ml PMA for 24 hours, followed by 24 hours in media prior to infection. Differentiated THP-1 macrophages were either infected with M. avium strains or M. abscessus strains for 1 hour with an multiplicity of infection (MOI) of 5 or uninfected. Cells were washed twice, followed by 1 hour of antibiotic treatment with 200 μg/ml of amikacin to remove extracellular bacteria. After the antibiotic step, cells were washed once more and BisEDT (BIZ) or amikacin was added once to selected wells (see in vitro doses below).

The treatments included: vehicle, amikacin, Soluble BisEDT (S Biz), Insoluble BisEDT (I Biz), and amikacin+BisEDT. The time points for sample preparation were 72 h for M. abscessus strains and 4- and 7-days post infection for M. avium strains. At the indicated timepoints cell media was removed and replaced with 400 ul of 0.1% triton x-100 in H₂O for lysis. Wells were pipetted 25 times to disrupt cells then diluted and plated for CFU enumeration.

### Results

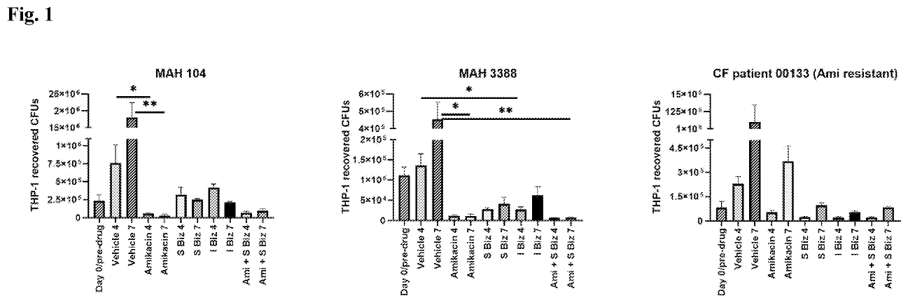

Using the MICs determined for each M. avium strain (see Table 3), M. avium survival was determined in THP-1 macrophages (FIG. 1). Infection CFUs were determined after 1 hour of infection and labeled as Day 0/pre-drug. As shown in FIG. 1 (first panel), the CFU count of untreated M. avium strain 104 (vehicle) grew over a 4-day or 7-day period. Treatment with amikacin greatly reduced the bacteria load in each case. Treatment of this same strain with soluble BisEDT (S Biz; DMSO solution) or insoluble BisEDT (I Biz) provided a bacteriostatic effect. When BisEDT was combined with amikacin, the bacterial load was substantially reduced at both time periods. A similar pattern was observed in M. avium strain 3388 (FIG. 1, second panel). However, in the CF patient strain 00133 that is resistant to amikacin, a bacteriostatic effect was observed, as each of the BisEDT formulations was effective in keeping the CFU count at levels similar to initial infection (FIG. 1, third panel).

TABLE 3
Treatment of M. avium-infected macrophages with BisEDT.
M. avium

| | amikacin | sol. | Insol. |
|---|---|---|---|
| Strain | (μg/mL) | BisEDT (μg/mL) | BisEDT (μg/mL) |
| 104 | 8 | 4 | 4 |
| 3388 | 8 | 4 | 4 |
| 1333 | 8 (resistant) | 2 | 8 |

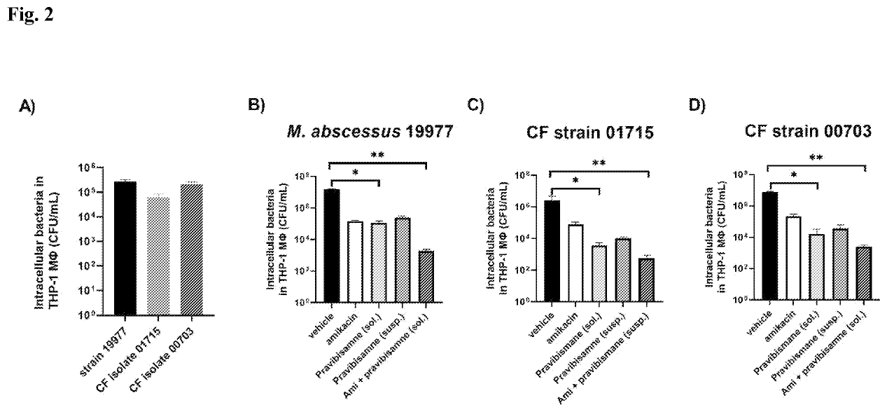

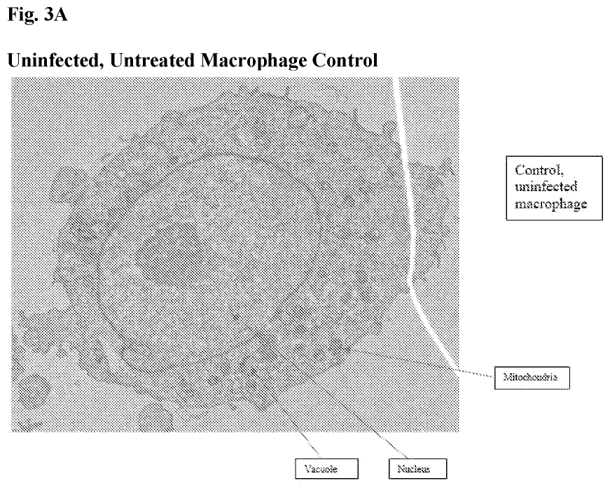

Using the MICs determined for each M. abscessus strain (see Table 4), M. abscessus survival was determined in THP-1 macrophages (FIG. 2). Infection CFUs were determined after 1 hour of infection and labeled as Day 0/pre-drug. After 3 days, the bacterial load of untreated M. abscessus strain 19977 increased inside of THP-1 cells. BisEDT reduced bacterial loads in THP-1 cells (FIG. 2, first panel). Amikacin treatment somewhat reduced CFU counts from initial infection rates, but the difference was not significant. However, as shown by the data, combining BisEDT with amikacin provided a synergistic affect as the bacterial CFU count in macrophages was reduced compared to when each drug was administered alone (FIG. 2, first panel). In both CF patient strains a similar pattern is obtained—amikacin treatment is bacteriostatic while treatment with BisEDT greatly reduced bacterial load in macrophages. Again, a synergistic effect was observed when BisEDT and amikacin are combined (FIG. 2, second and third panels).

TABLE 4
Treatment of M. abscessus-infected macrophages with BisEDT.
M. abscessus

| Strain | amikacin (μg/mL) | sol. EDT(μg/mL) | Insol. BisEDT (μg/mL) |
|---|---|---|---|
| 19977 | 32 | 1 | 1 |
| 01715 | 16 | 1 | 1 |
| 00703 | 64 | 4 | 2 |

### Conclusions—In Vitro Studies

BisEDT is bactericidal in macrophages infected with the three clinical isolates of M. avium tested, including an isolate of M. avium that is amikacin-resistant.

As shown in FIG. 1, for MAH 104, statistically significant differences were found between the vehicle 4 and amikacin 4 data and vehicle 7 and amikacin 7 data (p values of 0.0272 and 0.0017 respectively). For MAH 3388, statistically significant differences were found between the vehicle 4 data and the combination of amikacin and insoluble BisEDT 4, between the vehicle 7 data and the combination of amikacin and insoluble BisEDT 7, and the vehicle 7 and amikacin 7 data (p values of 0.0133, 0.0085, 0.0220 respectively). All analysis used a Kruskal Wallis test.

The effect of BisEDT on M. avium strains can therefore be augmented in combination with amikacin. Synergy was observed, as the effect of the combined treatment was greater than either compound used alone.

BisEDT is also very active in vitro against M. abscessus strains. THP-1 macrophages infected with clinically relevant strains of M. abscessus treated with BisEDT demonstrated a reduction in intracellular M. abscessus 3 days post-treatment.

As shown in FIG. 2, for MAB 19977, statistically significant differences were found between the vehicle and soluble BisEDT group and between the vehicle and amikacin+soluble BisEDT group (p values of 0.0370 and 0.0029 respectively). For CF Patient strain 1715, statistically significant differences were found between the vehicle and soluble BisEDT group and between the vehicle and amikacin+soluble BisEDT group (p values of 0.0295 and 0.0029 respectively). For CF Patient strain 007303, statistically significant differences were found between the vehicle and soluble BisEDT group and between the vehicle and amikacin+soluble BisEDT group (p values of 0.0466 and 0.0029 respectively) All analysis used a Kruskal Wallis test.

Thus, in the macrophage system, BisEDT was shown to be very active against M. abscessus alone and in combination with amikacin. BisEDT was bactericidal by itself (a 3-log reduction in M. abscessus level was observed in CF patient isolates) and the effect against M. abscessus was increased when combined with amikacin. The data suggest that BisEDT is capable of killing M. abscessus intracellularly.

### Example 4: BisEDT Mechanism of Action Against Intracellular NTMs

THP-1 macrophages were differentiated with 50 ng/ml PMA for 24 hours, followed by 24 hours in media prior to infection. Differentiated THP-1 macrophages were infected with M. avium or M. abscessus for 1 hour with a multiplicity of infection (MOI) of 10. Infected cells were washed twice, followed by 1 hour of antibiotic treatment with 200 μg/ml of amikacin to remove extracellular bacteria. After the antibiotic step, cells were washed once more and 4 μg/ml insoluble BisEDT (BIZ) was added to selected wells. The treatments included: (1) untreated, (2) untreated+infected with MAH 104, (3) untreated+infected with M. abscessus, (4) BIZ treated, (5) BIZ treated+infected with MAH 104, and (6) BIZ treated+infected with M. abscessus. The time points for sample preparation were 24 h and 48 h.p.i. Cells were not washed prior to transmission electron microscopy TEM sample preparation, to preserve any extracellular bacteria that may have been present. Cells were detached by treatment with 5 mM EDTA for 30 μmin, quenched with 1×HBSS, and suspended in fixative buffer with 2.5% glutaraldehyde, 1% formaldehyde, and 0.1 M sodium cacodylate for 24 hours prior to submission to the electron microscopy facility for processing. Samples were sectioned, dehydrated, and visualized by a FEIT Titan 80-200 TEM/STEM microscope.

### Results

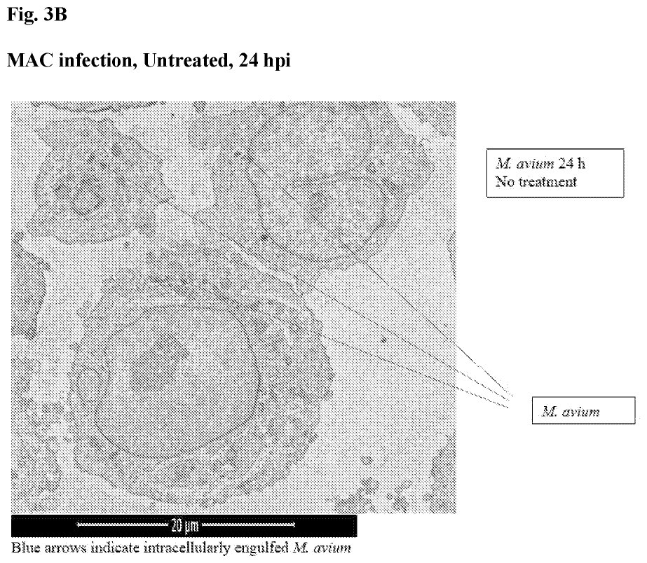

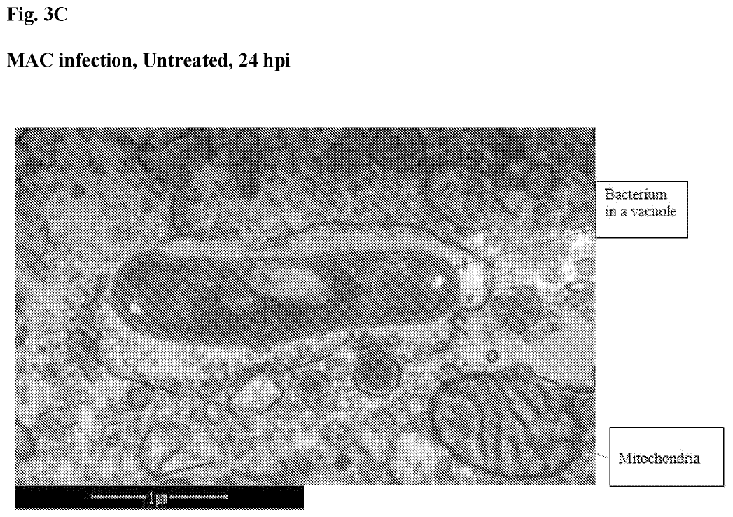

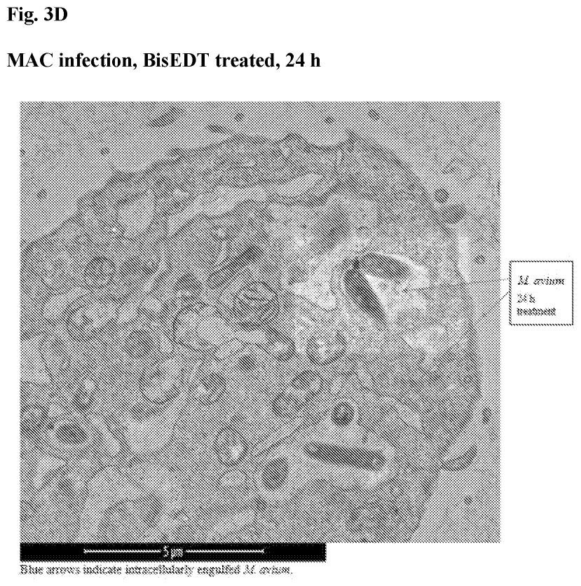

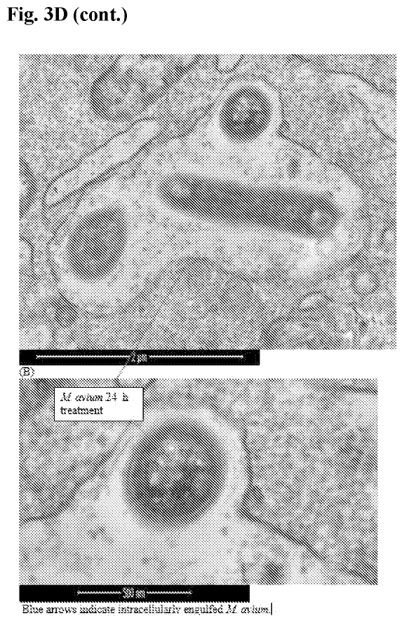

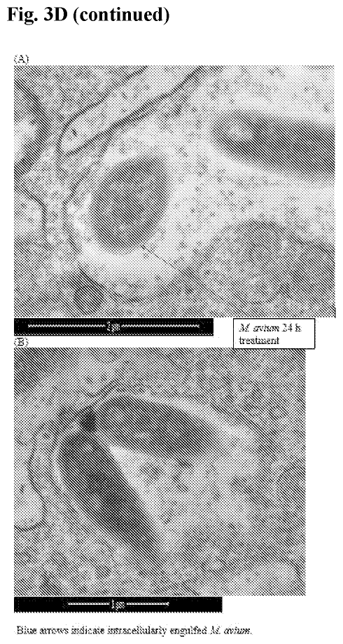

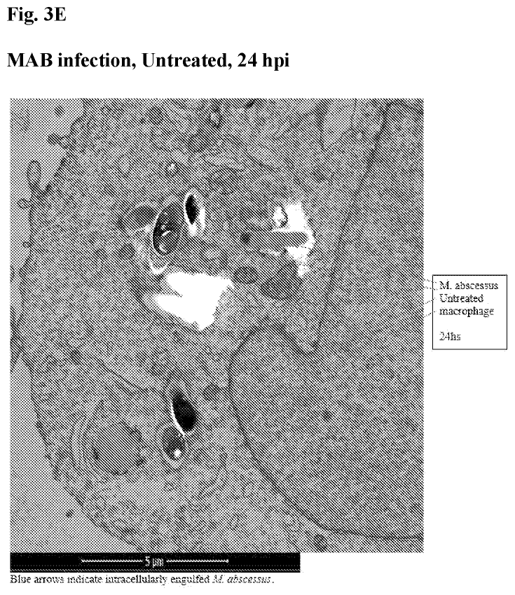

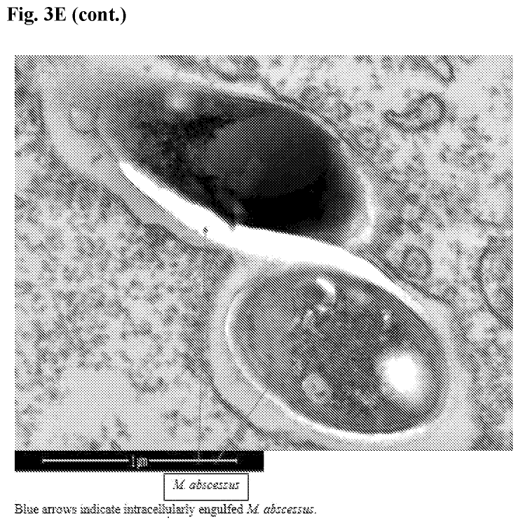

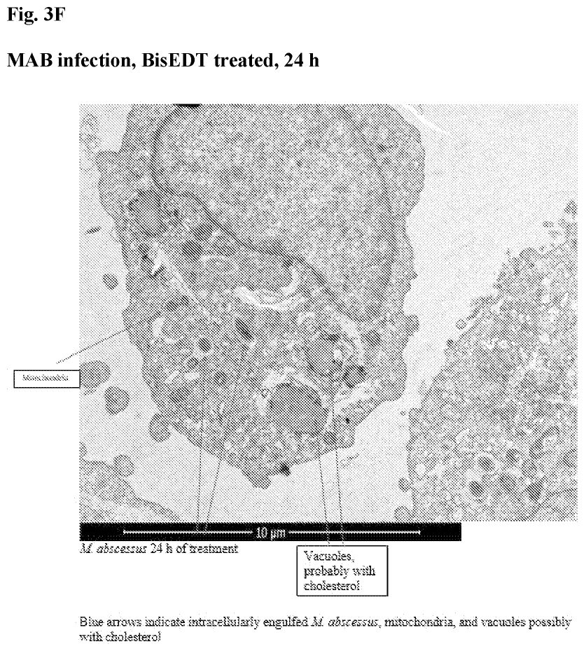

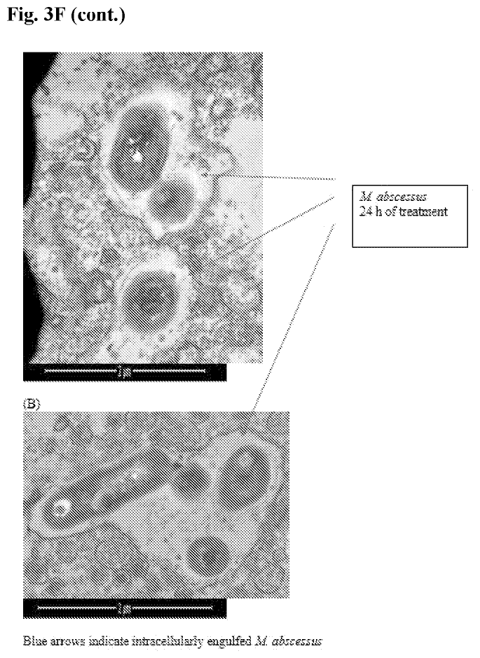

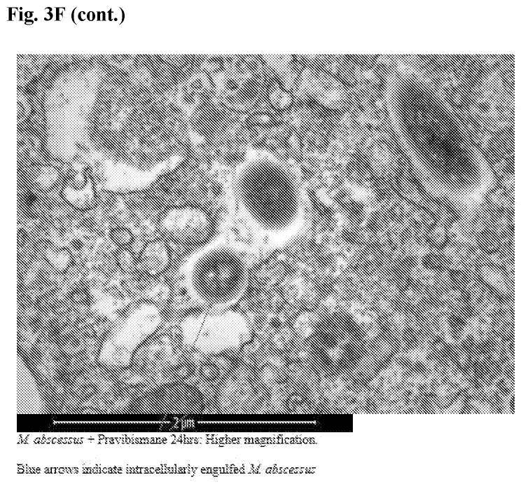

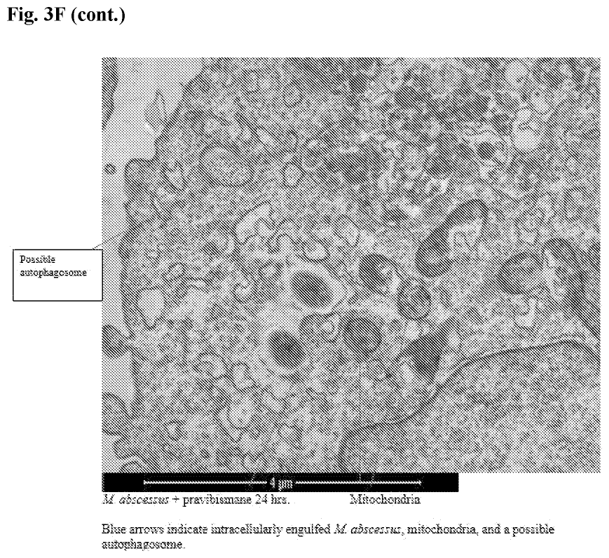

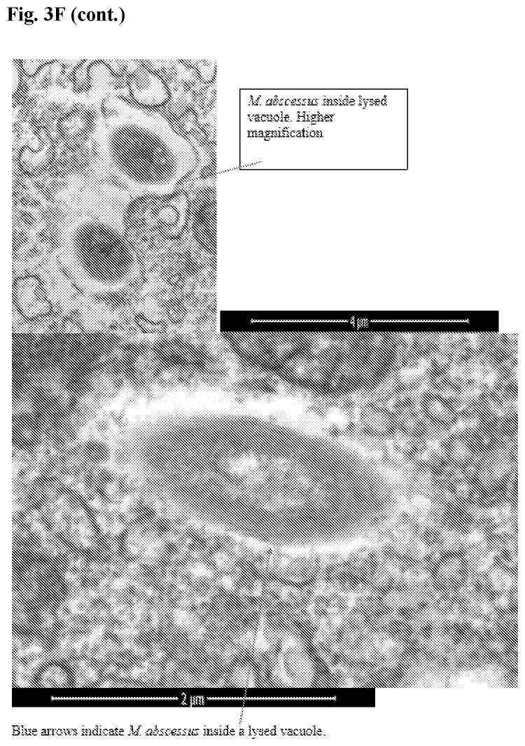

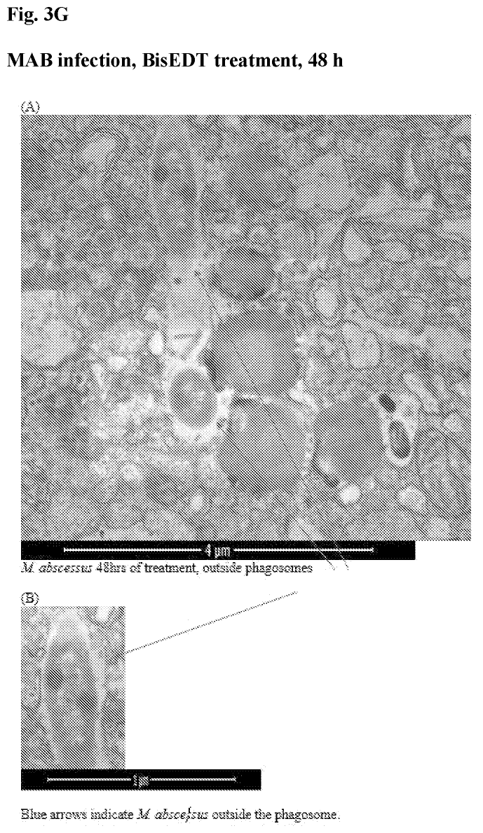

As shown in TEM images of FIGS. 3A-3F, BisEDT appears to act (primarily or secondarily) on the mycobacteria cell wall, as observed by the loss of the integrity of the polar region initially, and the whole cell wall in a more advanced stage. There are no major differences observed between the two species of bacteria after 2 d of macrophage infection. However, M. abscessus appears to leave the macrophage earlier than M. avium (3 d vs. 5 d). From an analysis of the images, it is believed the compounds get into eukaryotic cells by pinocytosis or through energy dependent transport mechanisms.

### Example 5: Efficacy of BisEDT Against Biofilm-Grown CF-Related Pathogens

### Background/Scope

The development of antibiotic-resistant bacterial strains has reduced the efficacy of antibiotics commonly used in the treatment of chronic infections in CF patients. Additionally, it has been shown that biofilms formed by many of these bacterial strains also impede the activity of these antibiotics. The identification and development of novel drugs that are effective both against drug-resistant bacterial strains and against biofilm is an urgent need for the treatment of chronic infections in CF patients.

The scope of this study is to evaluate the antibiofilm performance of BisEDT against the performance of the antibiotics currently used for the treatment of infections in CF patients, using a minimum biofilm eradication concentration (MBEC) assay.

### MBEC Assay

In this study the MBEC assay was followed as described in the Innovotech Procedural Manual, version 2.1 with minor modifications to assess the antibiofilm activity of the antimicrobial compounds under investigation. The MBEC value refers to the Minimum Biofilm Eradication Concentration of each antimicrobial for each strain. The MBEC value is defined as the minimum antibiotic concentration where the optical density (OD) value at recovery is less than 10% of the control OD value and there is no growth observed with visual examination of the wells.

### Strains

Several strains of eight microbial species were tested (Table 5A) with different sets of antimicrobials (Table 5B) at various concentrations to determine the MBEC value for each strain.

A new liquid culture was grown from a single colony in Mueller-Hinton II cation-adjusted (CAMHB) broth, up to the late log phase, for storage. Strains were stored in 15% glycerol stocks at −80° C.

TABLE 5A
Strains tested with modified MBEC assay.

| Species | Strains | | | | | | |
|---|---|---|---|---|---|---|---|
| P. aeruginosa | MR33 | [symbol] | MR60 | AGR2 | AGR9 | AGR13 | [symbol] |
| B. cepacia complex | [symbol] | SA114 | | | | | |
| S.aureus MRSA | SA35 | SA114 | | | | | |
| S. maltophilia | SM1 | SM2 | SM8 | SM9 | | | |
| Achromobacter spp. | AX12 | AX19 | | | | | |
| Acinstobacter baumannii | [symbol] | [symbol] | [symbol] | [symbol] | [symbol] | | |
| M. abscessus | MABS1 | MABS2 | MABS4 | MABS7 | MABS9 | MABS11 | |
| M. avium complex | 0119-8 | 0193-13 | ATCC 700808 | HMCO2 WO | HMCO2 RO | | |

[symbol]

indicates data missing or illegible when filed

TABLE 5B
Species and antibiotics tested.

| Species | Antibiotics | | | | | | | |
|---|---|---|---|---|---|---|---|---|
| P. aeruginosa | Meropenem | Celtazidime | Tobramycin | Amikacin | Aztreonam | Ciprofloxacin | Colistin | BisEDT |
| B. cepacia complex | Ceftazidime | Meropencon | Levofloxacin | BisEDT | | | | |
| S.aureus MRSA | Vancomycin | Clindamycin | Linezolid | BisEDT | | | | |
| S. maltophilia | Co-trimoxazok | Minocycline | Levofloxacin | BisEDT | | | | |
| Achromobacter spp. | Ceftazidime | Meropencom | Colistin | Tobramycin | BisEDT | | | |
| Acinstobacter baumannii | Ceftazitime | Meropenem | Levofloxacin | Amikacin | BisEDT | | | |
| M. abscessus | Imipenem | Clarithromycin | Amikacin | Cefoxitm | BisEDT | | | |
| M. avium complex | Rifampin | Clarithromycin | Amikacin | Ethambutol | BisEDT | | | |

### Procedure

Step 1: Inoculation. For resuscitation, all strains were streaked on TSA plates, except for the mycobacteria which were streaked on 7H10 media, from the frozen stocks, and grew overnight at 37° C. (Table 6). From the plate, one colony was picked for inoculation in organism specific medium (OSM) and grown in a shaker incubator at 37° C. at 225 RPM until mid-log phase (OD 0.2-0.8). At mid log phase the culture was diluted in OSM medium (Table 6) to an OD of 0.05. 150 μL of it were aliquoted in each corresponding well in a 96-well MBEC plate (inoculation plate). Subsequently, the MBEC plates were incubated static at 37° C. for the appropriate time for each species (Table 7). During incubation, biofilm formed on the pegs.

Step 2: Challenge. Following incubation, the peg lid with the biofilm was washed twice in PBS and placed on a second 96-well plate with 200 μl of antibiotic dilutions (challenge plate). The challenge plate was then incubated at 37° C. for 18 h to allow exposure of the biofilm to antibiotics.

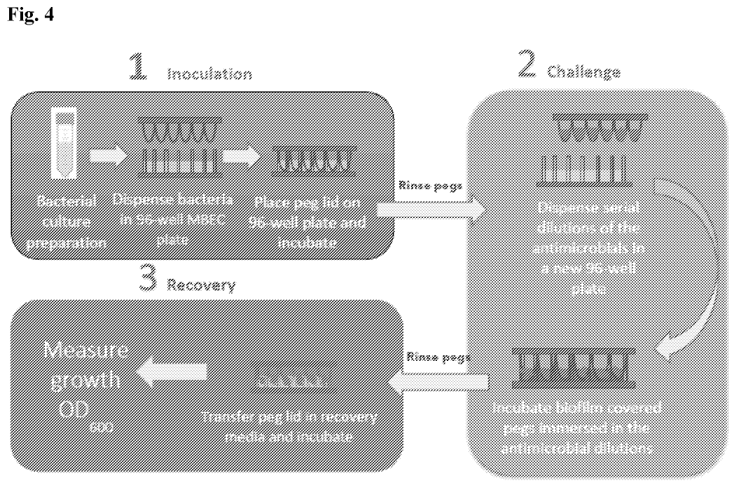

Step 3: Recovery. In the final step, the peg lid (with the biofilm after treatment with the antibiotics) was washed again twice in PBS and placed on a third 96-well plate (recovery plate) with 200 μl growth medium in each well to allow growth of any bacterial cells resistant to antibiotics at any concentration. The recovery of resistant cells was assessed by an end-point measurement of the optical density at 600 nm (OD600) for each well with a plate reader (FIG. 4).

TABLE 6
Organism specific media.

| | Media | | |
|---|---|---|---|
| Species | Resuscitation | Inoculum growth | Recovery |
| Mycobacterium abscessus | 7H10 | CAMHB | CAMHB |
| Mycobacterium avium complex | 7H10 | CAMHB + 10% OADC | N/A |

TABLE 7
Timeline of the three stages of the modified MBEC assay

| | Timeline | | |
|---|---|---|---|
| Species | Inoculation | Challenge | Recovery |
| Mycobacterium abscessus | 6-7 days | 18 h | 1-2 days |
| Mycobacterium avium complex | 14-21 days | N/A | N/A |

### Results

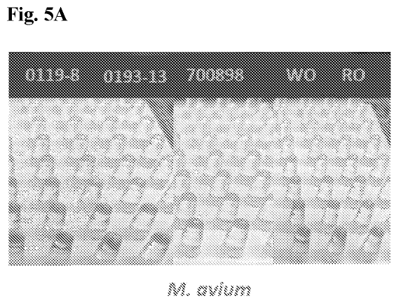

M. abscessus and M. avium strains tested with the antibiotics were evaluated for the formation of biofilms on the pegs of the MBEC plate. Biofilm formation was assessed with the crystal violet assay. In brief, the pegs were washed twice in PBS and subsequently they were immersed in a 96-well plate with 0.1% of crystal violet for 10 minutes. The pegs were then washed twice again with PBS and biofilm formation was indicated by the appearance of purple color (see FIGS. 5A-5B).

M. avium complex. Five strains of M avium complex were tested, but none formed biofilm on the pegs (FIG. 5A). Therefore, no further assessment of the antimicrobials antibiofilm activity was possible to be performed for these strains.

M. abscessus. Six clinical isolates of M. abscessus were tested, MABS1, MABS2, MABS4, MABS7, MABS9 and MABS11. All strains showed resistance to imipenem, cefoxitin, amikacin and clarithromycin at high concentrations apart from strain MABS4 that showed susceptibility to amikacin at a concentration of 8 μg/mL and to clarithromycin at a concentration of 4 μg/mL. Strains MABS1, MABS2, MABS9 and MABS11 also showed resistance to BisEDT at a concentration of 32 μg/mL, whereas strains MABS4 and MABS7 were susceptible to BisEDT at a concentration less or equal to 0.5 μg/mL.

TABLE 8
MIC and MBEC for M. abscessus strains.

| | | | Strain | | | | | |
|---|---|---|---|---|---|---|---|---|
| | Antibiotic | | MABS1 | MABS2 | MABS4 | MABS7 | MABS9 | MABS11 |
| Mycobacteria | Imipenem | MIC | 8 | 1 | 8 | 16 | 16 | 8 |
| abscessus | | MBEC | >32 | >16 | >64 | >64 | >64 | >64 |
| | Cefoxitin | MIC | 8 | 8 | 16 | 16 | 32 | 16 |
| | | MBEC | >128 | >128 | >128 | >128 | >256 | >256 |
| | Amikacin | MIC | 16 | 32 | 4 | 2 | 8 | 1 |
| | | MBEC | >128 | >512 | <8 | >16 | >64 | >16 |
| | Clarithromycin | MIC | 16 | 4 | 0.125 | 0.5 | 0.25 | 2 |
| | | MBEC | >32 | >32 | <=4 | >8 | >8 | >=32 |
| | BisEDT | MIC | 0.25 | 0.125 | 8 | 0.06 | 0.06 | 0.06 |
| | | MBEC | >32 | >32 | <=0.5 | <=0.5 | >32> | >32 |

To facilitate the comparison of BisEDT with antibiotics that have widely differing effective concentrations, we normalized MBEC values on a scale of 0-1, where 0 represents complete susceptibility and 1 represents complete resistance. To do this, the following formula was applied to each MBEC value together with the maximum and minimum concentration of the relevant antibiotic: ([MBEC]−[Ab]ₘᵢₙ)/([Ab]ₘₐₓ−[Ab]ₘᵢₙ)

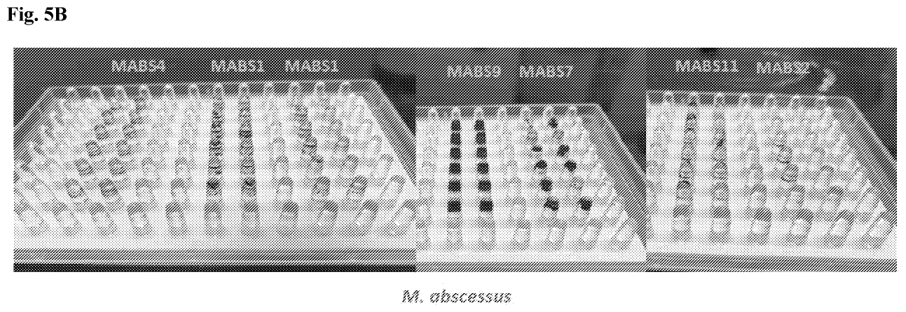

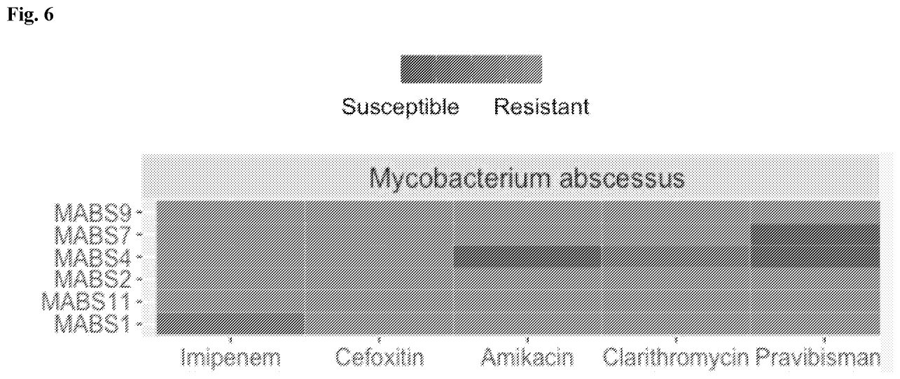

BisEDT was found to be effective relative to the comparator antibiotics for two strains of M. abscessus (MABS7 and MABS4). The evaluated isolates showed very little susceptibility to any of the comparator antibiotics (FIG. 6).

The M. abscessus data showed some variability among experimental replicates. This is possibly, in part, due to the variable clumping of the cells observed in the biofilm biomass on the pegs. The clumping of the cells also impacted the OD readings. Thus, visual examination of the plates was performed to deal with this challenge. For replicate experiments with variable results, the final MBEC values were determined based on the higher MBEC concentration observed among the replicates. Although four out of six M. abscessus strains showed resistance to BisEDT at concentrations higher than 32 μg/mL, the same strains also showed that at concentrations higher than 0.125 μg/mL their recovery was considerably slower compared to their recovery at concentrations lower than that, indicating that BisEDT impacts the growth of these strains at lower concentrations than their MBEC value.

None of the five M. avium strains that were tested formed biofilm, therefore antibiotic testing of the biofilm was not possible.

The effectiveness of BisEDT on the biofilm of multidrug resistant (MDR) M. abscessus strains makes it a promising next generation antibiotic candidate.

### Example 6: Efficacy of BisEDT Against Chronic NTM Infection in Mice

### Background

BisEDT (pravibismane) is a novel bioenergetic inhibitor antibiotic that affects the energy flow in the bacterial membrane which prevents ATP production. This mechanism of action is becoming increasingly popular for new antibiotics. Our purpose is to evaluate the efficacy of BisEDT through inhalation against pulmonary and M. abscessus infection in a mouse model. Since 2012, the collection of small molecule inhibitors of bioenergetics has expanded dramatically. The inhibitors have now become a major component (>30%) of all new antimycobacterial drugs in clinical trials and are included in more than 65% of Phase III trial regimens. Bioenergetic inhibitors have been minimally explored for efficacy in NTM infection and can act as an alternative for resistant NTM infections. These formulations can improve the outcome of patient clearance to previously resistant strains of NTM.

Inhalation distribution of antimicrobials is becoming the preferred application for lung infection treatment. The benefits of inhalation include site specific drug targeting which avoids systematic administration, higher therapeutic concentrations at the site of infection, and reduces off target effects and some toxicity. Earlier work on rats had shown high tolerability at doses as high as 59 μg/kg/day for the duration of 28 days without adverse effects on the animals. This research is aimed at identifying a new treatment for pulmonary NTM, which will minimize the discomfort and cost of care of affected individuals, and possibly also shorten the duration of the infection.

### Study Design

In vivo studies were conducted in mice to model chronic M. abscessus infection. The animals were treated for 28 days and grouped according to dosage: vehicle, low dose BisEDT, high dose BisEDT, and amikacin. At the end of treatment, blood, lungs and spleens were collected for analysis. Because BisEDT showed strong efficacy against M. abscessus in vivo, further work was done to investigate the propensity of the bacteria to develop resistance, both naturally and from strains collected from mouse lungs. Histopathology was conducted on lung samples from the in vivo studies.

M. abscessus Infection Protocol

A total of sixty female SCID/Beige mice between 6-8 weeks of age were used. Mice were placed up to 4 animals per cage and allowed 1 week of acclimation prior to infection.

Inoculums of M. abscessus strain 19977 (MAB) used in this study were made from highly virulent frozen stocks with low passage numbers. Bacteria were allowed to grow to log phase after 4 days on 7H10 media agar plates at 37° C. Once confluent, inoculums were made by suspending bacteria at 10⁹ in Hank's balanced salt solution (HBSS) as determined by optical density (O.D.) and further quantified by serial dilution to determine the CFU/mL of suspension. At this higher concentration of bacteria, only 20 μL of inoculum was necessary to deposit 10⁸ bacteria per mouse.

### Anesthetization

On the day of infection mice were anesthetized with isoflurane by the drop jar method. Mice were scruffed in a standard one-handed grip and the nose of the mouse is held of a tube containing gauze containing a small amount of isoflurane. Mice were monitored for breathing and once rapid breaths have slowed down initially the mouse is removed from direct anesthesia and 20 μL of bacterial suspension was deposited on the left nostril. The mice then slowly breath in the droplet. Each mouse was then held in hand until complete consciousness was obtained and then the mouse was replaced into its home cage. After infection was initiated, 3 weeks were allowed to pass in order to allow the bacteria to establish a chronic lung pathology for eventual treatment.

After establishing each infection, 12 mice were euthanized the day before treatment began to determine the baseline bacterial load. Mice were euthanized using CO₂ in accordance with the recommendations of the Guide for the Care and Use of Laboratory Animals (NRC 2011). Lungs and spleens were collected and homogenized then serial diluted for CFU enumeration.

### Drug Inhalation Protocol

A nose-only inhalation exposure chamber from CH technologies was used for this experiment. Mice were split into four treatment groups, Vehicle (buffer containing no BisEDT), amikacin (100 μg/kg/day), BisEDT “low” (200 μg/kg/day), and BisEDT “high” (1000 μg/kg/day). Each treatment group consisted of 12 mice. Ten of these mice were used for CFU determination and two mice are used for histology/pathology/lung pharmacokinetics. Treatment was conducted 6 days a week over the course of 28 days total. To create the correct level of deposited dose of each drug tested, 10 mL of test article were made. Amikacin in HBSS at 20 mg/mL was made and stored at 4° C. until needed. BisEDT “high” at a concentration of 8 mg/mL was made once a week and stored at room temperature. BisEDT “low” was diluted from the 8 mg/mL to 1.6 mg/mL the day of treatment. Each treatment used an LC star reusable nebulizer from Pari. Animals were restrained and placed on the inhalation apparatus. Respiratory hose was connected to the inhalation apparatus and a compressor was connected to the nebulizer. The rate of air flow from the compressor to the nebulizer remained at 8.5 L/minute, which generated a nebulized fog that each mouse breathed in over 45 μmin.

### Sample and Data Collection

After 28 days of treatment with BisEDT, animals were euthanized by Co₂ and secondary cardiac puncture to collect whole blood for quantitation of BisEDT concentration. Lungs and spleens from the CFU mice were homogenized with 1:1 H₂O:Dey-Engley neutralizing broth to deactivate BisEDT. CFUs were determined after serial dilution. Mice for histology and the lung pair split, one half in formalin the other half weighed and snap frozen in liquid N₂ to determine tissue concentration of BisEDT.

Spleens were also deposited in formalin. Samples for histology were processed by the Veterinary Diagnostic Laboratory. Slides were stained with hematoxylin-eosin (HE stain) for general pathology as well as a slide for acid-fast stain to highlight the mycobacterial infection in lung tissue.

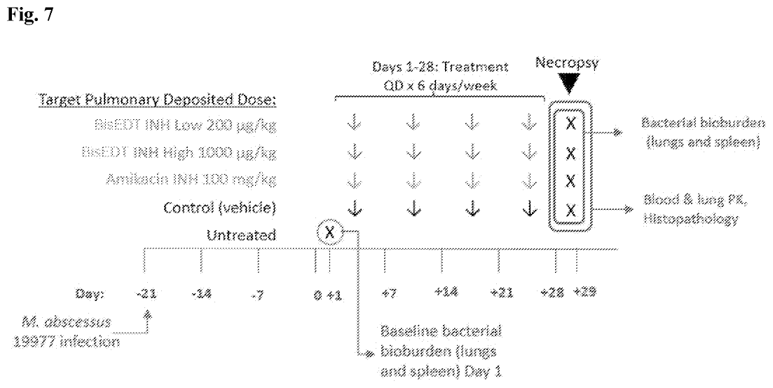

The dosage groups and study schedule for the M. abscessus in vivo study are summarized in Table 9 and FIG. 7.

TABLE 9
M. abscessus infection in SCID mice study overview.

| | | | | | | | Mice | | | |
|---|---|---|---|---|---|---|---|---|---|---|
| | | | Target | Target | | | | Day 1 | | |
| | | Inflation | Presented | Pulmonary | Formulation | Volume/ | | Baseline | Day 29 | Day 29 |
| | Test | Dosing | Dose | Deposited | Conc. | Delivery | Female | CFU | CFU | Lung & Blood |
| Group | Substance | Regimen | (mg/kg/day) | Dose | (mg/mL) | Time | ( [symbol] ) | ( [symbol] ) | ( [symbol] ) | [symbol] |
| 1 | [symbol] | [symbol] | [symbol] | [symbol] | [symbol] | [symbol] | [symbol] | [symbol] | [symbol] | [symbol] |
| 2 | [symbol] | [symbol] | [symbol] | [symbol] | [symbol] | [symbol] | [symbol] | [symbol] | [symbol] | [symbol] |
| 3 | [symbol] | [symbol] | [symbol] | [symbol] | [symbol] | [symbol] | [symbol] | [symbol] | [symbol] | [symbol] |
| 4 | [symbol] | [symbol] | [symbol] | [symbol] | [symbol] | [symbol] | [symbol] | [symbol] | [symbol] | [symbol] |
| 5 | [symbol] | [symbol] | [symbol] | [symbol] | [symbol] | [symbol] | [symbol] | [symbol] | [symbol] | [symbol] |

N/A = Not applicable
ns = Not sampled [symbol]
indicates data missing or illegible when filed

### Results of M. Abscessus Chronic Lung Infection Study

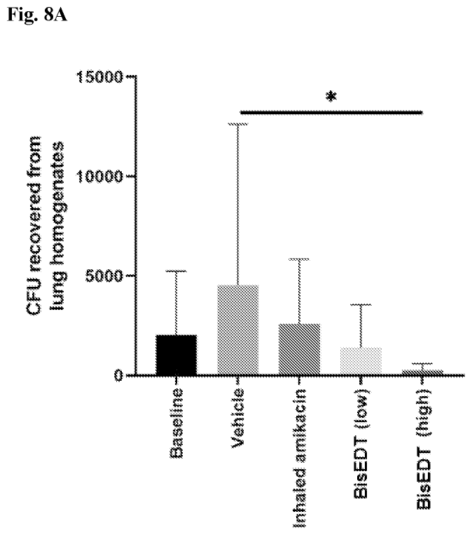

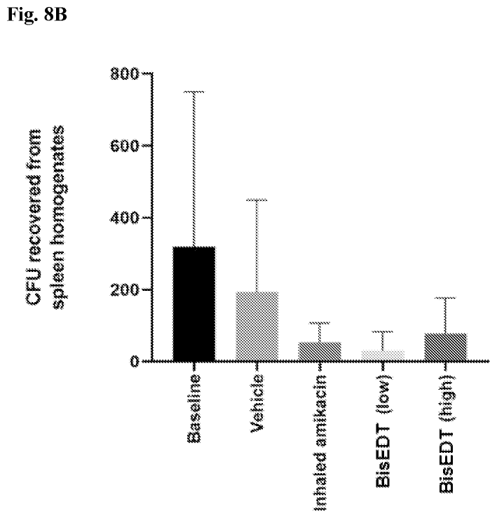

BisEDT doses were well tolerated by the mice and the mice were responsive and within the normal weight range at the end of treatment. Data for CFU recovery from lung and spleen homogenates is provided in FIGS. 8A and 8B, respectively. Using a Kruskal-Wallis test, the lung CFU data showed a dose-dependent response to BisEDT, as well as a statistically significant difference (p-value=0.0163) between the high-dose group and the vehicle control.

As shown in FIG. 8A by the reduction of CFU count in lung homogenates, administration of 200 μg/kg of BisEDT (low) in mice infected with M. abscessus provided a bacteriostatic effect. BisEDT is bactericidal at 1000 μg/kg (high) delivered to the lungs of mice infected with against M. abscessus. In the macrophage system it was also very active (bactericidal) against all three clinical strains tested. Moreover, the activity against M. abscessus in this mouse model suggests that BisEDT is active against M. abscessus in biofilm phenotype.

### Histopathology

Following inhalation exposure to aerosol atmospheres of either vehicle or BisEDT for 28 days, as described above, lungs and spleens were removed from vehicle-treated and antibiotic-treated mice. Mice for histology and lung tissue samples were selected prospectively. Each had their respective lung pair split. One-half was fixed in formalin pending further histologic processing, while the other half was weighed and frozen pending bioanalysis for quantitation of BisEDT in lung tissue. Spleens were also fixed in formalin prior to further processing.

Samples for histology were processed. Slides of tissue-sections were prepared and stained with hematoxylin-eosin (HE stain) for general pathology, while in each case a duplicate slide was prepared for acid-fast stain to highlight the mycobacterial infection in lung tissue.

### Example 7: Evaluation of Resistance to BisEDT in Mouse Lung Homogenates

BisEDT showed in vivo efficacy against M. abscessus, so studies were conducted to examine the propensity of M. abscessus to develop resistance to the drug product. The first study sought to show whether bacteria that had survived inhalation treatment had developed resistance: bacteria from treated lung homogenates were exposed to MIC concentrations to evaluate growth.

Several samples appeared to show growth at the previously determined MIC level, typically indicating the development of resistance after treatment. In this case, however, confirmatory MIC work showed that the MIC of the bacteria had not significantly changed and M. abscessus had not developed resistance over the course of treatment. A second study sought to determine the concentration of bacteria required for natural resistance to emerge at bactericidal concentrations of BisEDT.

Lung homogenates from the M. abscessus μmouse infection were thawed from frozen at 37° C. for 2 hours. To reduce the effect of the Dey-Engley deactivation buffer, mouse homogenates were diluted 1:10 in water before replating samples onto 7H10 agar plates containing 1 μg/ml BisEDT (MIC level). Once plated samples were incubated at 37° C. over 3 days and plate colonies were counted to determine CFUs of resistance.

Colony forming units were compared from the initial plating with no BisEDT in the agar plates; to replating on agar plates containing 1 μg/mL BisEDT. The percent of resistance determined is provided in Table 10.

TABLE 10
Resistance development in exposed lung
homogenate treated with BisEDT.

| | Mouse | | Percent |
|---|---|---|---|
| | homogenate | Resistance | resistant |
| | (CFU) | plating (CFU) | (%) |
| BisEDT | 6.70E+03 | 6.00E+02 | 9.0 |
| (200 μg/kg/day) | 3.70E+02 | 0.00E+00 | 0 |
| | 2.50E+03 | 7.00E+02 | 28 |
| | 2.40E+02 | 1.00E+02 | 41.7 |
| | 8.60E+02 | 1.00E+02 | 11.6 |
| | 2.00E+01 | 0.00E+00 | 0 |
| | 0.00E+00 | 0.00E+00 | 0 |
| | 3.00E+03 | 1.10E+03 | 36.7 |
| | 6.00E+01 | 1.00E+02 | 166.7 |
| | 2.40E+02 | 0.00E+00 | 0 |
| BisEDT | 1.00E+01 | 0.00E+00 | 0 |
| (1000 | 4.00E+01 | 0.00E+00 | 0 |
| μg/kg/day) | 0.00E+00 | 0.00E+00 | 0 |
| | 1.30E+02 | 0.00E+00 | 0 |
| | 9.60E+02 | 1.00E+02 | 10.4 |
| | 7.50E+02 | 1.00E+02 | 13.3 |
| | 0.00E+00 | 0.00E+00 | 0 |
| | 2.40E+02 | 1.00E+02 | 41.7 |
| | 4.00E+01 | 0.00E+00 | 0 |
| | 3.50E+02 | 0.00E+00 | 0 |

Colony forming units were compared from the initial plating with no BisEDT in the agar plates to replating on agar plates containing 1 μg/mL BisEDT. The ratio is expressed as a percentage in Table 10 above, and appeared to show the development of resistance to BisEDT.

To confirm whether observable colonies were truly resistant to BisEDT and not simply the result of deactivation media still present within the lung homogenates or artifacts of the distribution of BisEDT on the agar plates, randomly selected isolates were grown on 7H10 from multiple mice to reach confluency, and then a standard MIC analysis was performed as described above. As shown in Table 11, IC levels from this study were determined to be 0.5 μg/mL. Previous MIC levels for BisEDT were determined to be 1 μg/mL. Generally, to be considered a significant change in resistance, at least 2 IC levels of difference should be observed. Thus, this difference is not considered a change in resistance. The MBC levels of these select isolates also did not show a change in resistance.

Conclusion: There is little development of resistance seen in the chronic M. abscessus study among mice treated with BisEDT for 28 days.

TABLE 11
MIC of select bacterial isolates from murine lung homogenates.

| | BisEDT (μg/mL) | | | | | | | | | | |
|---|---|---|---|---|---|---|---|---|---|---|---|
| Mouse ID | 16 | 8 | 4 | 2 | 1 | 0.5 | 0.25 | 0.125 | 0 | 7H9 only | Blank |
| C1L | 0.127 | 0.122 | 0.128 | 0.117 | 0.119 | 0.118 | 0.129 | 0.305 | 0.774 | 0.107 | 0.089 |
| C3L | 0.136 | 0.143 | 0.141 | 0.138 | 0.14 | 0.137 | 0.359 | 1.142 | 1.477 | 0.113 | 0.088 |
| C4L | 0.129 | 0.13 | 0.127 | 0.131 | 0.126 | 0.121 | 0.142 | 0.349 | 0.519 | 0.105 | 0.09 |

### Example 8. Evaluation of the Emergence of Resistance in M. abscessus to BisEDT Treatment

Emergence of resistance is determined by using the bactericidal concentration of BisEDT and differing amounts of M. abscessus bacteria. Using this procedure determines the spontaneous development of resistance in a suspension of bacteria.

M. abscessus was grown to log phase on 7H10 agar at 37° C. for 3 days. From this plate, a bacterial suspension of 10¹¹ was made in PBS-T (phosphate buffered saline containing 0.05% tween 20). Ten tubes of 7H9 broth (900 μL each) containing 4 μg/mL of BisEDT or 7H9 broth alone were made and 100 μL of high bacterial suspension was added to one tube then serially diluted into each other tube (1:10). After bacterial suspension was added and thoroughly mixed, tubes were incubated at 37° C. in a shaking incubator at 200 rpm for 3 days to allow for the antibiotic to affect the bacteria. Optical density determined levels of bacteria growth by taking 100 mL of bacteria suspension and placed in a flat bottom 96-well plate for reading on the Epoch spectrophotometer at 595 nm.

TABLE 12
Spontaneous development of resistance to BisEDT in M. abscessus.

| | Bacteria concentration (CFU/mL) | | | | | | | | | | |
|---|---|---|---|---|---|---|---|---|---|---|---|
| | 10¹⁰ | 10⁹ | 10⁸ | 10⁷ | 10⁶ | 10⁵ | 10⁴ | 10³ | 10² | 10¹ | Blank |
| BisEDT^{a} | 0.485 | 0.107 | 0.099 | 0.101 | 0.096 | 0.094 | 0.097 | 0.096 | 0.096 | 0.094 | 0.084 |
| 7H9 broth | 0.681 | 0.679 | 0.709 | 0.744 | 0.793 | 0.714 | 0.826 | 0.513 | 0.377 | 0.372 | 0.083 |
| only | | | | | | | | | | | |

^{a}4 μg/mL BisEDT in 7H9 culture broth

### Results

At the MBC, or bactericidal concentration of BisEDT (4 μg/mL), MAB was only able to overcome the antibiotic at 10¹⁰ bacteria as determined by optical density (Table 12). This bacterial growth can be compared to the same amounts of bacteria grown in just 7H9 broth without antibiotic. MAB grew easily in the 7H9 broth and was reflected in the optical density above.

Conclusion: Incubation in the presence of a bactericidal concentration of BisEDT (4 μg/mL) demonstrated that natural resistance to the compound only occurs when the bacterial inoculum was 10¹⁰ or above. This means it takes a high concentration of bacteria to be present in order for the M. abscessus to spontaneously become resistant to this level of BisEDT.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

What is claimed is:

1. A method of treating an infection in a subject caused by nontuberculous Mycobacterium (NTM), the method comprising administering to the subject an effective amount of a bismuth-thiol (BT) composition that comprises a BT compound.

2. The method of claim 1, wherein the infection is a pulmonary infection.

3. The method of claim 1, wherein the infection is an extrapulmonary infection.

4. The method of any one of claims 1-3, wherein the NTM infection is caused by an antibiotic-resistant strain of NTM.

5. The method of any one of claims 1-4, wherein the NTM infection is caused by M. avium, M. avium subsp. hominissuis (MAH), M. abscessus, M. chelonae, M. bolletii, M. kansasii, M. ulcerans, M. avium complex (MAC) (M. avium and M. intracellulare), M. conspicuum, M. kansasii, M. peregrinum, M. immunogenum, M. xenopi, M. marinum, M. malmoense, M. marinum, M. mucogenicum, M. nonchromogenicum, M. scrofulaceum, M. simiae, M. smegmatis, M. szulgai, M. terrae, M. terrae complex, M. haemophilum, M. genavense, M. asiaticum, M. shimoidei, M. gordonae, M. nonchromogenicum, M. triplex, M. lentiflavum, M. celatum, M. fortuitum, M. fortuitum complex (M. fortuitum and M. chelonae), or a combination thereof.

6. The method of any one of claims 1-4, wherein the NTM infection is caused by M. abscessus, M. avium, or a combination thereof.

7. The method of any one of claims 1-4, wherein the NTM lung infection is caused by M. avium complex (M. avium and M. intracellulare).

8. The method of any one of claims 5-7, wherein the M. avium is M. avium subsp. hominissuis.

9. The method of any one of claims 1-8, wherein the NTM infection is a biofilm-associated NTM infection.

10. The method of any one of claims 1, 2 and 4-9, wherein the NTM infection is a chronic pulmonary infection.

11. The method of any one of claims 1-10, wherein the NTM infection is located in or on the lung mucosa, the bronchi, the alveoli, the macrophages, and/or the bronchioles.

12. The method of any one of claims 1-11, wherein the NTM infection is at least partially located in the macrophages.

13. The method of claim 11 or 12, wherein the macrophages are THP-1 macrophages.

14. The method of any one of claims 11-13, wherein upon administration of the BT composition to the subject, the bacterial load in the macrophages is reduced.

15. The method of any one of claims 11-14, wherein the macrophages are infected with one or more strains of M. abscessus and/or M. avium.

16. The method of any one of claims 1-15, wherein the NTM infection is resistant to treatment with amikacin.

17. The method of any one of claims 1-16, wherein the NTM infection is resistant to macrolide or azalide therapy.

18. The method of any one of claims 1-17, wherein the subject has a chronic lung condition.

19. The method of claim 18, wherein the chronic lung condition is the chronic lung condition is cystic fibrosis, chronic bronchitis, emphysema, bronchiectasis, pulmonary fibrosis, asbestosis, pneumonitis, chronic obstructive pulmonary disorder (COPD), or asthma.

20. The method of claim 18 or 19, wherein the chronic lung condition is cystic fibrosis.

21. The method of any one of claims 1-20, wherein the subject is administered about 30 μg to about 3,000 μg of BT compound per dose.

22. The method of claim 21, wherein the subject is administered about 100 μg to about 1,000 μg of BT compound per dose.

23. The method of any one of claims 1-22, wherein the BT composition is administered once per month, twice per month, three times per month, four times per month, once every two weeks, once per week, twice per week, or three times per week.

24. The method of any one of claims 1-23, wherein the BT composition is administered once or twice daily.

25. The method of any one of claims 1-24, wherein the BT compound is administered to the lungs of the subject.

26. The method of any one of claims 1-25, wherein the BT compound is administered by inhalation.

27. The method of any one of claims 1-26, wherein the BT composition is administered for a period of less than 24 months, less than 18 months, less than 12 months, less than 9 months, less than 6 months, less than 3 months, or less than 1 month.

28. The method of any one of claims 1-27, wherein the BT composition is administered for a period of 1 day to 56 days.

29. The method of any one of claims 1-27, wherein the BT composition is administered for a period of 14 days to 28 days.

30. The method of any one of claims 25-29, wherein the concentration of bismuth in the lungs after a single daily dose is from about 0.03 μg/g lung tissue to about 3 μg/g lung tissue.

31. The method of any one of claims 25-29, wherein the concentration of bismuth in the lungs after 28 daily doses is from about 0.3 μg/g lung tissue to about 60 μg/g lung tissue.

32. The method of any one of claims 1-31, wherein the BT compound is selected from BisBAL, BisEDT, Bis-dimercaprol, BisDTT, Bis-2-mercaptoethanol, BisDTE, BisPyr, BisEry, BisTol, BisBDT, BisPDT, BisPyr/BAL, BisPyr/BDT, BisPyr/EDT, BisPyr/PDT, BisPyr/Tol, BisPyr/Ery, bismuth-1-mercapto-2-propanol, BisEDT/CSTMN (1:1), BisPyr/CSTMN (1:1), BisBAL/CSTMN (1:1), BisTOL/CSTMN (1:1), and BisEDT/2-hydroxy-1-propanethiol.

33. The method of claim 32, wherein the BT compound is selected from BisEDT, BisBAL, BisPyr, BisEry, BisTol, BisBDT, or BisEDT/2-hydroxy-1-propane thiol.

34. The method of claim 32, wherein the BT compound is BisEDT or BisBAL.

35. The method of claim 32, wherein the BT compound is BisEDT.

36. The method of any one of claims 1-35, further comprising administering an effective amount of amikacin, clarithromycin, azithromycin, ethambutol, rifampicin, tigecycline, linezolid, imipenem, cefoxitin, or combination thereof to the subject in need.
